(19) United States
(12) Patent Application Publication
(10) Pub. No.: US 2023/0332243 A1
(43) Pub. Date: Oct. 19, 2023

(54) PATIENT SELECTION BIOMARKERS FOR TREATMENT WITH ULK INHIBITORS

(71) Applicant: Salk Institute for Biological Studies, La Jolla, CA (US)

(72) Inventors: Reuben J. SHAW, San Diego, CA (US); Sonja N. BRUN, San Diego, CA (US); Lilian EICHNER, Del Mar, CA (US)

(21) Appl. No.: 18/028,751

(22) PCT Filed: Sep. 30, 2021
(86) PCT No.: PCT/US2021/052927

§ 371 Date: Mar. 27, 2023

Related U.S. Application Data
(60) Provisional application No. 63/085,917, filed on Sep. 30, 2020.

(51) Int. Cl. C12Q 1/6886; A61K 31/505

(52) U.S. Cl. CPC C12Q 1/6886; A61K 31/505; C12Q 2600/106; C12Q 2600/158

(57) ABSTRACT

Provided herein are biomarkers and method of selecting patients for treating diseases, including cancer, with ULK inhibitors using the biomarkers.

### CROSS-REFERENCE TO RELATED APPLICATIONS

This application claims priority benefit of the filing date of U.S. Provisional Patent Application Ser. No. 63/085,917, filed on Sep. 30, 2020, the disclosure of which is incorporated by reference herein in its entirety.

### BACKGROUND

Autophagy is a central cellular mechanism for elimination of damaged proteins, protein complexes, and organelles. This conserved process plays crucial roles in the cellular response to nutrient deprivation and other stresses, in addition to being required for proper cellular and tissue homeostasis during embryonic development and in defense against pathogens. Defects in autophagy pathways are associated with certain human pathologies, including infectious diseases, neurodegenerative disorders, and cancer. In spite of these highly conserved fundamental cellular functions, the molecular and biochemical details of how autophagy is initiated for different cargoes, and the coordination of steps starting from autophagosome initiation to ultimate fusion with the lysosome remain poorly understood.

### SUMMARY

Provided herein are biomarkers for patient selection for treatment with ULK inhibitors and methods of selecting patients for treatment with ULK inhibitors using the biomarkers. In some embodiments, the inhibitors inhibit ULK1. In some embodiments, the inhibitors are specific for ULK1. In some embodiments, the inhibitors inhibit both ULK1 and ULK2. In some instances, the inhibitors provided herein are useful for the treatment of various diseases, including cancer.

Disclosed herein are methods of treating cancer in a subject in need thereof by administering to the subject a therapeutically effective amount of a ULK inhibitor, wherein the cancer in the subject has a distinct expression of at least one of biomarker gene in Table 1 or Table 2. Also disclosed herein are methods of predicting a likelihood of success of treating a cancer with a ULK inhibitor in a subject in need thereof by obtaining a gene expression profile of a plurality of genes from a tissue of the subject, wherein the plurality of genes comprises at least one gene in Table 1 or at least one gene in Table 2, and predicting the likelihood of success of a ULK inhibitor treatment based on the gene profile. Also disclosed herein are methods of selecting a subject for a ULK inhibitor treatment against a cancer in the patient by obtaining a gene expression profile of a plurality of genes from a tissue of the subject, wherein the plurality of genes comprises at least one gene in Table 1 or at least one gene in Table 2, and selecting the subject for the ULK inhibitor treatment based on the gene profile.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a tumor growth inhibition graph of 35 patients derived from xenograft models.

FIG. 2 shows a heatmap of hierarchical clustering of genes differentially expressed in extreme responders and non-responders.

FIG. 3A shows a heatmap of cluster 8 genes differentially expressed in extreme responders and non-responders.

FIG. 3B shows a heatmap of cluster 4 genes differentially expressed extreme responders and non-responders.

FIG. 3C shows a heatmap of cluster 1 genes differentially expressed extreme responders and non-responders.

FIG. 4A shows a heatmap of cluster 3 genes differentially expressed in extreme responders and non-responders.

FIG. 4B shows a heatmap of cluster 7 or cluster 6 genes differentially expressed in extreme responders and non-responders.

FIG. 4C shows a heatmap of cluster 11 genes differentially expressed in extreme responders and non-responders.

FIG. 5A shows a heatmap of cluster 10 genes differentially expressed in extreme responders and non-responders.

FIG. 5B shows a heatmap of cluster 5 genes differentially expressed in extreme responders and non-responders.

FIG. 5C shows a heatmap of cluster 9 genes differentially expressed in extreme responders and non-responders.

FIG. 6A shows a heatmap of cluster 15 genes differentially expressed in extreme responders and non-responders.

FIG. 6B shows a heatmap of cluster 16 genes differentially expressed in extreme responders and non-responders.

FIG. 7A shows a heatmap of cluster 12 or cluster 13 genes differentially expressed in extreme responders and non-responders.

FIG. 7B shows a heatmap of cluster 14 genes differentially expressed in extreme responders and non-responders.

FIG. 8A shows a heatmap of 9 gene signatures from original analysis without full cohort of tumors.

FIG. 8B shows a heatmap of 9 gene signatures of clusters with all models.

FIG. 9 shows a heatmap of cluster 1 genes.

FIG. 10 shows a heatmap of cluster 2 genes.

FIG. 11 shows a heatmap of cluster 3 genes.

FIG. 12 shows a heatmap of cluster 4 genes.

FIG. 13 shows a heatmap of cluster 5 genes.

FIG. 14 shows a heatmap of cluster 6 genes.

FIG. 15 shows a heatmap of cluster 7 genes.

FIG. 16 shows a heatmap of cluster 8 genes.

FIG. 17 shows a heatmap of cluster 9 genes.

FIG. 18 shows a heatmap of cluster 10 genes.

FIG. 19 shows a heatmap of cluster 11 genes.

FIG. 20 shows a heatmap of cluster 12 genes.

FIG. 21 shows a heatmap of cluster 13 genes.

FIG. 22 shows a heatmap of cluster 14 genes.

FIG. 23 shows a heatmap of cluster 15 genes.

FIG. 24 shows a heatmap of cluster 16 genes.

FIG. 25 shows a heatmap of cluster 17 genes.

### DETAILED DESCRIPTION

Provided herein are methods of treating a disease with a ULK inhibitor as a monotherapy. Also provided herein are methods of treating a disease with a ULK inhibitor and an additional therapeutic agent. Further provided herein are compounds useful as ULK inhibitors. In some instances, the ULK inhibitor is a ULK1 specific inhibitor. In some instances, the ULK inhibitor inhibits both ULK1 and ULK2.

### Autophagy

In certain instances, autophagy is a cellular response to loss of nutrients in which cells catabolize various proteins and organelles to provide building blocks and critical metabolites needed for cell survival. In some instances, autophagy plays an important homeostatic role in many tissues by removing protein aggregates and defective organelles that accumulate with cellular damage over time. While genetics first defined the core components of autophagy conserved across all eukaryotes, the molecular details of how the different autophagy complexes regulate one another, and the precise temporal and spatial ordering of biochemical events involved in autophagy induction are typically considered to be poorly understood currently.

In healthy individuals, normal autophagy is, in certain instances, an important process for balancing sources of energy at critical times in development and in response to nutrient stress. In certain instances, autophagy also plays a housekeeping role in removing misfolded or aggregated proteins, clearing damaged organelles, such as mitochondria, endoplasmic reticulum and peroxisomes, as well as eliminating intracellular pathogens. Thus, autophagy is often thought of as a survival mechanism. In various instances, autophagy is either non-selective or selective in the removal of specific organelles, ribosomes and protein aggregates. In addition to elimination of intracellular aggregates and damaged organelles, in certain instances, autophagy promotes cellular senescence and cell surface antigen presentation, protects against genome instability and prevents or inhibits necrosis, giving it an important role in preventing, treating, or inhibiting diseases such as cancer, neurodegeneration, cardiomyopathy, diabetes, liver disease, autoimmune diseases and infections.

In some instances, defects in autophagy pathways are associated with a number of human pathologies, including infectious diseases, neurodegenerative disorders, and cancer. In some instances, the role of autophagy differs in different stages of cancer development; for example, in some instances, initially, autophagy has a preventive effect against cancer, but once a tumor develops, the cancer cells, in certain instances, utilize autophagy for their own cytoprotection. In some cancers, the mutations that cause uncontrolled cell growth which results in the formation of tumors or other cancerous tissue also effectuates changes in autophagy. In some instances, these changes in the autophagic pathways in the cancer cells results in increased survivability and durability of cancer cells. In some instances, this leads to the cells resisting apoptosis and cell death in response to standard cancer treatments, thus reducing the efficacy of cancer therapeutics. In certain instances, rather than killing the cancer cells, the therapeutics merely have the effect of arresting cancer tissue growth, with the cancer tissue entering a cystostatic phase upon treatment. Consequently, in some instances, the cancerous tissue is not killed during treatment, the growth is simply arrested. Upon cessation of treatment, the cancerous tissue is able to resume growth, thus increasing symptoms and complications for the patient. In light of this, in some instances, the addition of a therapeutic that disrupts autophagy has the effect of converting the cytostatic response of the cancer cells to cancer cell death.

In certain cancers, the changes in autophagy caused by the cancer are important for the survival of the cancer cells. As the mutations that cause cancer result in uncontrolled cell growth, in some instances, these cells rely on autophagy to properly regulate the consumption of nutrients to ensure the survival of the cells in conditions that would cause the death of a healthy cell. Thus, methods of inhibiting autophagy in cells present, in certain instances, a method of treating cancer without the need of an additional cancer therapeutic.

### ULK1 and ULK2

In many instances, ULK1 and/or ULK2 are important proteins in regulating autophagy in mammalian cells. In certain instances, ULK1 and/or ULK2 are activated under conditions of nutrient deprivation by several upstream signals, which is followed by the initiation of autophagy. The requirement for ULK1 and/or ULK2 in autophagy initiation has been studied in the context of nutrient deprivation.

In certain instances, ULK1 complex, combining ULK1, ATG (autophagy-related protein) 13 (ATG13), FIP200 (focal adhesion kinase family interacting protein of 200 kDa), and ATG101 is one of the first protein complexes that comes in to play in the initiation and formation of autophagosomes when an autophagic response is initiated. Additionally, ULK1 is considered to be unique as a core conserved component of the autophagy pathway which is a serine/threonine kinase, making it a particularly unique target of opportunity for development of compounds to control autophagy. Equally importantly for a clinical therapeutic index for agents inhibiting ULK1, mice genetically engineered to completely lack ULK1 are viable without significant pathology. Thus, in many instances, a ULK1 selective kinase inhibitor is well tolerated by normal tissues, but not by tumor cells that have become reliant on ULK1-mediated autophagy for survival.

In some instances, ULK2 takes over the functional role of ULK1 when ULK1 function has been inhibited. Thus, in some cases, an inhibitor that is effective for both ULK1 and ULK2 is desirable to mitigate this effect.

### ULK Inhibitors

In some instances, ULK inhibitors include any ULK inhibitors disclosed in WO2016/033100A1 titled “Novel Ulk1 Inhibitors And Methods Using Same”, disclosed in PCT Publication No. WO2021/163627, titled “Non-Macrocyclic ULK 1 Inhibitors”, disclosed in PCT Publication No. WO2021163629, titled “Non-Macrocyclic ULK 1 Inhibitors”, or disclosed in PCT Publication No. WO2021/163633, titled “Mono- And Combo-Therapies With ULK1 Inhibitors”, the disclosures of which are each incorporated by reference herein in their entireties.

In certain embodiments, the ULK inhibitor is at least one selected from the group consisting of a 2-(substituted)amino-4-(substituted)amino-5-halo-pyrimidine; 2-(substituted)amino-4-(substituted) amino-5-(halo)alkyl-pyrimidine; 2-(substituted)amino-4-(substituted)oxo-5-halo-pyrimidine; 2-(substituted)amino-4-(substituted)oxo-5-(halo)alkyl-pyrimidine; 2-(substituted)amino-4-(substituted)thio-5-halo-pyrimidine; and 2-(substituted)amino-4-(substituted)thio-5-(halo)alkyl-pyrimidine; or a pharmaceutically acceptable salt thereof.

Also disclosed herein are ULK inhibitors, or pharmaceutically acceptable salts thereof, having a structure of:

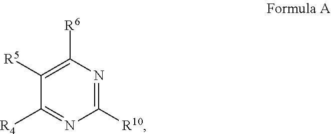

wherein in Formula A:
R¹⁰ is selected from the group consisting of: halogen; —OR¹¹ wherein R¹¹ is H, optionally substituted aryl, or optionally substituted heteroaryl; —NR¹R¹ wherein R¹ and R² are each individually selected from the group consisting of H, optionally substituted aryl, optionally substituted heteroaryl, optionally substituted cycloalkyl, and optionally substituted alkyl, or NR¹R² together form a heterocycle; or R⁴ and R¹⁰ together form a cyclic structure;
R⁴ is selected from the group consisting of optionally substituted amino, optionally substituted aryloxy, optionally substituted heteroaryloxy, optionally substituted alkoxy, N-heterocyclic, optionally substituted thiol, optionally substituted alkyl, hydroxyl and halogen;
R⁵ is selected from the group consisting of H, hydroxyl, optionally substituted alkyl, halo, optionally substituted alkoxy, or optionally substituted aryl, optionally substituted carboxyl, cyano, and nitro, or R⁵ and R⁶ together form a cyclic structure; and
   R⁶ is H, halogen, or haloalkyl.

In some embodiments, R¹⁰ is —OR¹¹. In some embodiments, R¹¹ is optionally substituted aryl or optionally substituted heteroaryl. In some embodiments, R¹¹ is an optionally substituted phenyl ring fused with a 5- or 6-membered cycloalkyl, hetercycloalkyl, aryl, or heteroaryl ring, wherein the 5- or 6-membered ring is independently optionally substituted. In some embodiments, R¹¹ is optionally substituted napthyl, optionally substituted tetrahydronapthyl, optionally substituted quinolyl, optionally substituted indolyl, or optionally substituted tetrahydroquinolyl. In some embodiments, R¹¹ is optionally substituted napthyl, optionally substituted tetrahydronapthyl, optionally substituted quinolyl, optionally substituted indolyl, or optionally substituted tetrahydroquinolyl, wherein the napthyl, tetrahydronapthyl, quinolyl, indolyl, or tetrahydroquinolyl is optionally substituted with —OH, —NH₂, alkyl, halogen, or alkoxy. In some embodiments, R¹¹ is napthyl optionally substituted with —OH, —NH₂, alkyl, halogen, or alkoxy. R¹¹ is unsubstituted napthyl, unsubstituted tetrahydronapthyl, unsubstituted quinolyl, unsubstituted indolyl, or unsubstituted tetrahydroquinolyl. In some embodiments, R¹¹ is optionally substituted phenyl. In some embodiments, R¹¹ is phenyl optionally substituted with —OH, —NH₂, alkyl, halogen, or alkoxy.

In some embodiments, R¹⁰ is —NR¹R². In some embodiments, R¹ and R² together form a heterocycle. R¹ and R² together form an unsubstituted 4-8 membered heterocycle.

In some embodiments, R¹ is H or —C₁-C₆ alkyl. In some embodiments, R¹ is H or —CH₃. In some embodiments, R¹ is H.

In some embodiments, R² is optionally substituted alkyl or optionally substituted cycloalkyl. In some embodiments, R² is optionally substituted alkyl. In some embodiments, R² is optionally substituted cycloalkyl. In some embodiments, R² is unsubstituted cycloalkyl. In some embodiments, R² is cyclopropyl, cyclobutyl, or cyclopentyl. In some embodiments, R² is unsubstituted cyclopropyl, unsubstituted cyclobutyl, or unsubstituted cyclopentyl.

In some embodiments, R² is optionally substituted aryl or heteroaryl. R² is optionally substituted phenyl. In some embodiments, R² is phenyl optionally substituted with one or more substituents selected from alkyl, alkoxy, haloalkoxy, halogen, —S-alkyl, phenoxy, hydroxy, morpholinyl. R² is alkoxy substituted phenyl. R² is optionally substituted heteroaryl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl, wherein the pyridyl, pyrazinyl, pyrmidinyl, pyridazinyl, indolyl, benzimidazolyl, benzotriazolyl, or 7-azaindolyl is optionally substituted with one more substituent selected from —OH, —NH₂, alkyl, halogen, or alkoxy. In some embodiments, R² is optionally substituted 5- or 6-membered heteroaryl. In some embodiments, R² is an optionally substituted fused heteroaryl. In some embodiments, R² is an optionally substituted bicyclic fused ring system that contains at least one nitrogen atom. In some embodiments, R² is selected from the group consisting of

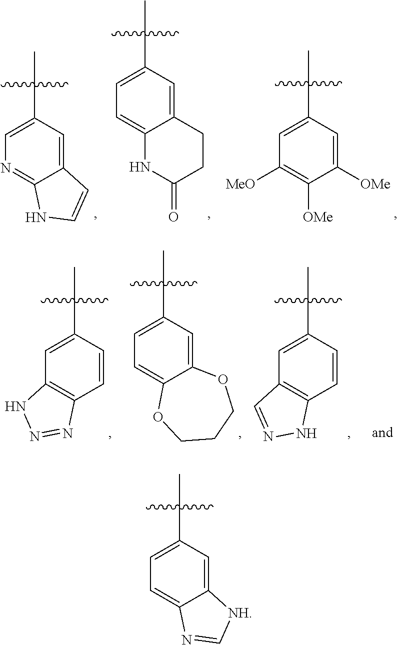

In some embodiments, R⁴ is selected from the group consisting of optionally substituted amino, optionally substituted aryloxy, optionally substituted heteroaryloxy, and optionally substituted alkoxy.

In some embodiments, R⁴ is optionally substituted aryloxy or optionally substituted heteroaryloxy. In some embodiments, R⁴ is aryloxy or heteroarylxy, wherein the aryloxy or heteroaryloxy is optionally substituted with one or more substituents selected from —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl. In some embodiments, R⁴ is selected from the group consisting of phenoxy, (C₁-C₆)alkoxy, and —O—(N-alkylbenzamide), particularly —O—(N—(C₁-C₆)alkylbenzamide). In some embodiments, R⁴ is

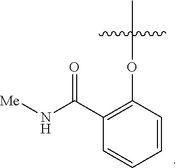 [SMILES: *Oc1ccccc1C(=O)NC]

In some embodiments, R⁴ is —S(C₁-C₆)alkyl, —O(C₁-C₆ alkyl), or —O(C₃-C₈ cycloalkyl). In some embodiments, R⁴ is —S(C₁-C₆)alkyl. In some embodiments, R⁴ is —O(C₁-C₆ alkyl). In some embodiments, R⁴ is —O(C₁-C₆ alkyl).

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ and R⁸ together form an unsubstituted 4-8 membered heterocycle. In some embodiments, R⁷ and R⁸ together form a heterocycle.

In some embodiments, R⁷ and R⁸ are each independently selected from H and C₁-C₆ alkyl with one or two substituents selected from —OH, —OMe, —C(═O)OMe, —C(═O)OH, —NH₂, —NHMe, —N(Me)₂, —NHCH₂CH₂OH, and cyclopropyl.

In some embodiments, R⁷ is H or —CH₃. In some embodiments, R⁷ is H.

In some embodiments, R⁸ is optionally substituted aryl or optionally substituted heteroaryl. In some embodiments, R⁸ is optionally substituted phenyl or optionally substituted pyridyl. In some embodiments, R⁸ is optionally substituted phenyl or optionally substituted pyridyl, wherein the phenyl or pyridyl is optionally substituted with —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl.

In some embodiments, R⁸ is phenyl optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is phenyl optionally substituted with —C(═O)NH(C₁-C₆ alkyl), alkoxy, or halogen. In some embodiments, R⁸ is phenyl optionally substituted with —C(═O)NHMe or —OMe.

In some embodiments, R⁸ is pyridyl is optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is pyridyl optionally substituted with —C(═O)NH(C₁-C₆ alkyl), alkoxy, or halogen. In some embodiments, R⁸ is pyridyl optionally substituted with —C(═O)NHMe or —OMe.

In some embodiments, R⁸ is cycloalkyl. In some embodiments, R⁸ is optionally substituted C₃-C₈ cycloalkyl. In some embodiments, R⁸ is unsubstituted C₃-C₈ cycloalkyl. In some embodiments, R⁸ is unsubstituted C₃-C₆ cycloalkyl. In some embodiments, R⁸ is cyclopropyl or cyclobutyl.

In some embodiments, R⁵ is H, halogen, C₁-C₃ fluroalkyl, or cyano. In some embodiments, R⁵ is Br, Cl, or —CF₃. In some embodiments, R⁵ is Cl. In some embodiments, R⁵ is Br. In some embodiments, R⁵ is —CF₃.

In some embodiments, R⁶ is H, —CF₃, or F. In some embodiments, R⁶ is H or F. In some embodiments, R⁶ is H. In some embodiments, R⁶ is F.

Also disclosed herein are ULK inhibitors or pharmaceutically acceptable salts thereof, having a structure of:

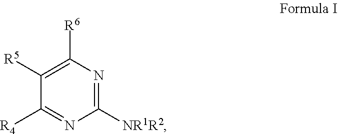

wherein in Formula I;
R¹ and R² are each individually selected from the group consisting of H, optionally substituted aryl, optionally substituted heteroaryl, optionally substituted cycloalkyl, and optionally substituted alkyl, or NR¹R² together form a heterocycle;
R⁴ is selected from the group consisting of optionally substituted amino, optionally substituted aryloxy, optionally substituted heteroaryloxy, optionally substituted alkoxy, N-heterocyclic, optionally substituted thiol, and optionally substituted alkyl;
R⁵ is selected from the group consisting of H, hydroxyl, optionally substituted alkyl, halo, optionally substituted alkoxy, and optionally substituted aryl; and
R⁶ is H or fluorine; or a pharmaceutically acceptable salt thereof.

In some embodiments, R¹ and R² together form a heterocycle. R¹ and R² together form an unsubstituted 4-8 membered heterocycle.

In some embodiments, R¹ is H or —C₁-C₆ alkyl. In some embodiments, R¹ is H or —CH₃. In some embodiments, R¹ is H.

In some embodiments, R² is optionally substituted alkyl or optionally substituted cycloalkyl. In some embodiments, R² is optionally substituted alkyl. In some embodiments, R² is optionally substituted cycloalkyl. In some embodiments, R² is unsubstituted cycloalkyl. In some embodiments, R² is cyclopropyl, cyclobutyl, or cyclopentyl. In some embodiments, R² is unsubstituted cyclopropyl, unsubstituted cyclobutyl, or unsubstituted cyclopentyl.

In some embodiments, R² is optionally substituted aryl or heteroaryl. R² is optionally substituted phenyl. In some embodiments, R² is phenyl optionally substituted with one or more substituents selected from alkyl, alkoxy, haloalkoxy, halogen, —S-alkyl, phenoxy, hydroxy, morpholinyl. R² is alkoxy substituted phenyl. R² is optionally substituted heteroaryl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl. In some embodiments, R² is optionally substituted pyridyl, optionally substituted pyrazinyl, optionally substituted pyrimidinyl, optionally substituted pyridazinyl, optionally substituted indolyl, optionally substituted benzimdazolyl, optionally substituted benzotriazolyl, or optionally substituted 7-azaindolyl, wherein the pyridyl, pyrazinyl, pyrmidinyl, pyridazinyl, indolyl, benzimidazolyl, benzotriazolyl, or 7-azaindolyl is optionally substituted with one more substituent selected from —OH, —NH₂, alkyl, halogen, or alkoxy. In some embodiments, R² is optionally substituted 5- or 6-membered heteroaryl. In some embodiments, R² is an optionally substituted fused heteroaryl. In some embodiments, R² is an optionally substituted bicyclic fused ring system that contains at least one nitrogen atom. In some embodiments, R² is selected from the group consisting of

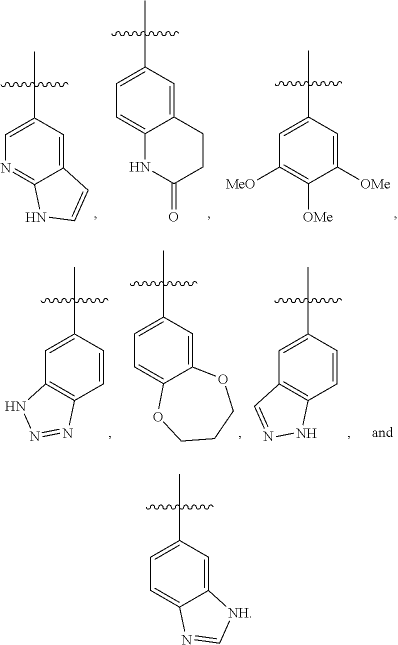

In some embodiments, R⁴ is selected from the group consisting of optionally substituted amino, optionally substituted aryloxy, optionally substituted heteroaryloxy, and optionally substituted alkoxy.

In some embodiments, R⁴ is optionally substituted aryloxy or optionally substituted heteroaryloxy. In some embodiments, R⁴ is aryloxy or heteroarylxy, wherein the aryloxy or heteroaryloxy is optionally substituted with one or more substituents selected from —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl. In some embodiments, R⁴ is selected from the group consisting of phenoxy, (C₁-C₆)alkoxy, and —O—(N-alkylbenzamide), particularly —O—(N—(C₁-C₆)alkylbenzamide). In some embodiments, R⁴ is

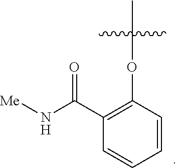 [SMILES: *Oc1ccccc1C(=O)NC]

In some embodiments, R⁴ is —S(C₁-C₆)alkyl, —O(C₁-C₆ alkyl), or —O(C₃-C₈ cycloalkyl). In some embodiments, R⁴ is —S(C₁-C₆)alkyl. In some embodiments, R⁴ is —O(C₁-C₆ alkyl). In some embodiments, R⁴ is —O(C₁-C₆ alkyl).

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ and R⁸ together form an unsubstituted 4-8 membered heterocycle. In some embodiments, R⁷ and R⁸ together form a heterocycle.

In some embodiments, R⁷ and R⁸ are each independently selected from H and C₁-C₆ alkyl with one or two substituents selected from —OH, —OMe, —C(═O)OMe, —C(═O)OH, —NH₂, —NHMe, —N(Me)₂, —NHCH₂CH₂OH, and cyclopropyl.

In some embodiments, R⁷ is H or —CH₃. In some embodiments, R⁷ is H.

In some embodiments, R⁸ is optionally substituted aryl or optionally substituted heteroaryl. In some embodiments, R⁸ is optionally substituted phenyl or optionally substituted pyridyl. In some embodiments, R⁸ is optionally substituted phenyl or optionally substituted pyridyl, wherein the phenyl or pyridyl is optionally substituted with —C(═O)NH(C₁-C₆ alkyl), alkoxy, halogen, —NH₂, NH(C₁-C₆ alkyl), —NH—[(C═O)C₁-C₆ alkyl], nitrile, —S—C₁-C₆ alykl, morpholino, C₁-C₆ alkyl, —SO₂—(C₁-C₆ alkyl), or haloalkyl.

In some embodiments, R⁸ is phenyl optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is phenyl optionally substituted with —C(═O)NH(C₁-C₆ alkyl), alkoxy, or halogen. In some embodiments, R⁸ is phenyl optionally substituted with —C(═O)NHMe or —OMe.

In some embodiments, R⁸ is pyridyl is optionally substituted with —C(═O)NH(C₁-C₆ alkyl), alkoxy, halogen, —NH₂, NH(C₁-C₆ alkyl), —NH(C═O)C₁-C₆ alkyl, nitrile, —S—C₁-C₆ alkyl, morpholinyl, C₁-C₆ alkyl, —SO₂—(C₁-C₆ alkyl), or haloalkyl. In some embodiments, R⁸ is pyridyl optionally substituted with —C(═O)NH(C₁-C₆ alkyl), alkoxy, or halogen. In some embodiments, R⁸ is pyridyl optionally substituted with —C(═O)NHMe or —OMe.

In some embodiments, R⁸ is cycloalkyl. In some embodiments, R⁸ is optionally substituted C₃-C₈ cycloalkyl. In some embodiments, R⁸ is unsubstituted C₃-C₈ cycloalkyl. In some embodiments, R⁸ is unsubstituted C₃-C₆ cycloalkyl. In some embodiments, R⁸ is cyclopropyl or cyclobutyl.

In some embodiments, R⁵ is H, halogen, C₁-C₃ fluroalkyl, or cyano. In some embodiments, R⁵ is Br, Cl, or —CF₃. In some embodiments, R⁵ is Cl. In some embodiments, R⁵ is Br. In some embodiments, R⁵ is —CF₃.

In some embodiments, R⁶ is H, —CF₃, or F. In some embodiments, R⁶ is H or F. In some embodiments, R⁶ is H. In some embodiments, R⁶ is F.

In some embodiments, R¹ is H and R² is not H. In other embodiments, R¹ is H and R² is an optionally substituted fused heteroaryl or an optionally substituted aryl. The optionally substituted fused heteroaryl, for example, may be a bicyclic fused ring system that include at least one nitrogen heteroatom. In some embodiments, R¹ is H and R² is an optionally substituted bicyclic fused ring system that includes at least one heteroatom. In some embodiments, R¹ is H and R² is an optionally substituted bicyclic fused ring system that includes at least one nitrogen heteroatoms. In some embodiments, R¹ is H and R² is an optionally substituted bicyclic fused ring system that includes at least two nitrogen heteroatoms. In some embodiments, R¹ is H and R² is an optionally substituted bicyclic fused ring system that includes at least two oxygen heteroatoms. The optionally substituted aryl, for example, may be a substituted or unsubstituted phenyl. The phenyl, for example, may be substituted with at least one alkoxy, preferably (C₁-C₆)alkoxy.

In some embodiments, R¹ is H and R² is selected from the group consisting of:

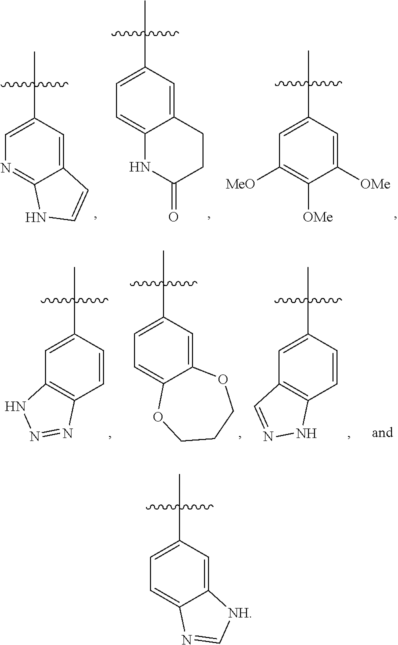

In some embodiments, R⁴ is selected from the group consisting of optionally substituted amino, optionally substituted aryloxy, optionally substituted heteroaryloxy, and optionally substituted alkoxy.

In some embodiments, R⁴ is selected from the group consisting of optionally substituted aryloxy, optionally substituted heteroaryloxy, and optionally substituted alkoxy. In particular embodiments, R⁴ is selected from the group consisting of optionally substituted phenoxy and optionally substituted alkoxy. In particular embodiments, R⁴ is selected from the group consisting of phenoxy, (C₁-C₆)alkoxy, and —O—(N-alkylbenzamide), particularly —O—(N—(C₁-C₆)alkylbenzamide). In particular embodiments, R⁴ is

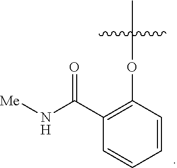 [SMILES: *Oc1ccccc1C(=O)NC]

In some embodiments, R⁴ is —NR⁷R⁸, wherein R⁷ and R⁸ are each individually selected from the group consisting of H, optionally substituted aryl, optionally substituted heteroaryl, cycloalkyl, and optionally substituted alkyl, or NR⁷R⁸ together form a heterocycle. In some embodiments, R⁷ is H and R⁸ is N-alkylbenzamide, particularly N—(C₁-C₆)alkylbenzamide. In some embodiments, R⁷ is H and R⁸ is phenyl. In some embodiments, R⁷ is H and R⁸ is alkoxy-substituted phenyl, particularly (C₁-C₆)alkoxy. In some embodiments, R⁷ is H and R⁸ is cyclopropyl. In some embodiments, R⁷ is H and R⁸ is cyclobutyl. In some embodiments, R⁷ is H and R⁸ is alkoxyalkyl, particularly (C₁-C₆)alkoxy(C₁-C₆)alkyl. In some embodiments, R⁷ is H and R⁸ is haloalkyl. In some embodiments, R⁷ is H and R⁸ is optionally substituted acyl. In some embodiments, R⁴ is —NH₂. In some embodiments, R⁴—OH.

In some embodiments, R⁵ is haloalkyl, particularly —CF₃. In some embodiments, R⁵ is Br. In some embodiments, R⁵ is Cl.

In some embodiments, R² is a fused heteroaryl ring and R⁴ is —NR⁷R⁸, wherein R⁷ is H and R⁸ is a fused heteroaryl ring. In particular embodiments, R² is selected from the group consisting of:

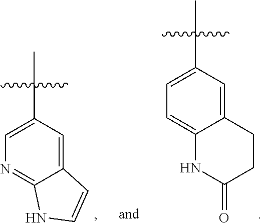 [SMILES: *c1cnc2[nH]ccc2c1 ; *c1ccc2c(c1)CCC(=O)N2]

In particular embodiments, R⁸ is:

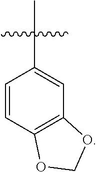 [SMILES: *c1ccc2c(c1)OCO2]

In some embodiments, R¹ is H or —CH₃, R² is alkoxy substituted phenyl; R⁴ is —NR⁷R⁸, wherein, R⁷ is H or —CH₃ and R⁸ is R⁸ is phenyl optionally substituted with —C(═O)NHMe or —OMe; R⁵ is Br, Cl, or —CF₃, and R⁶ is H or F.

In some embodiments, R¹ is H or —CH₃, R²is selected from the group consisting of

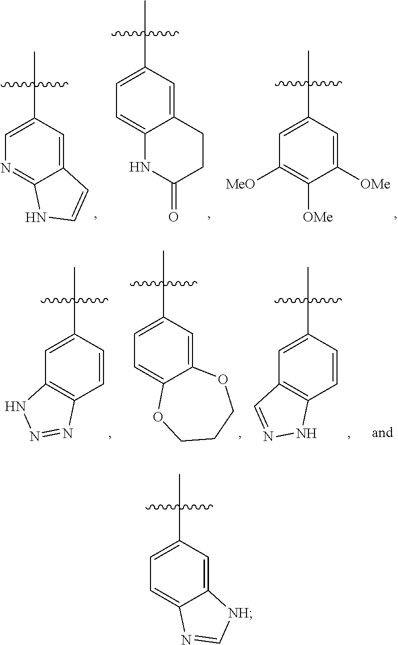

R⁴ is —NR⁷R⁸ wherein R⁷ is H or —CH₃ and R⁸ is phenyl optionally substituted with —C(═O)NHMe or —OMe; R⁵ is Br, Cl, or —CF₃, and R⁶ is H or F.

Any combination of the groups described above for the various variables is contemplated herein. Throughout the specification, groups and substituents thereof are chosen by one skilled in the field to provide stable moieties and compounds.

In certain instances, ULK inhibitors are efficacious as a monotherapy. In other instances, it is also surprising that ULK inhibitors are used/useful in augmenting or improving standard of care therapies. In some instances, the standard of care therapies do not involve mTOR inhibitors. In some instances, the cancer and ULK-mediated disorders do not implicate mTOR. In some instances, the ULK inhibitor inhibits ULK1. In some instances, the ULK inhibitor is a ULK1 specific inhibitor. In some instances, the ULK inhibitor inhibits both ULK1 and ULK2.

### ULK Inhibitor Treatment and Cancer

In some embodiments, the ULK inhibitor is administered alone to treat a disease or disorder as a monotherapy. In some embodiments, the ULK inhibitor is administered to the subject with an additional therapeutic agent. Details of methods of treatments are described in PCT Publication No. WO WO2021/163633, titled “Mono- And Combo-Therapies With ULK1 Inhibitors”, the disclosure of which is incorporated by reference herein in its entirety.

In some embodiments, the disease or disorder is characterized by abnormal autophagy. In some embodiments, the disease or disorder is characterized by abnormal ULK1 activity or expression (e.g., cancer). In some embodiments, the abnormal autophagy is therapeutically induced. In some embodiments, the disease or disorder is refractory. In some embodiments, the disease or disorder is refractory to treatment with an additional therapeutic agent. In embodiments, the disease or disorder is resistant to treatment with an additional therapeutic agent. In some embodiments, the additional therapeutic agent is a standard of care therapy.

In some embodiments, the disease or disorder treated with a ULK inhibitor is cancer. In some embodiments, the cancer is lung cancer, breast cancer, or pancreatic cancer. In some embodiments, the cancer is refractory. In some embodiments, the cancer is refractory to a standard of care therapy.

In some embodiments, the cancer is lung cancer. In specific embodiments, the lung cancer is non-small cell lung cancer. In some embodiments, the cancer is an advanced stage non-small cell lung cancer. In some embodiments, the cancer comprises a tumor. In some embodiments, the non-small cell lung cancer comprises a tumor. In some embodiments, the non-small cell lung cancer is characterized by abnormal autophagy. In some embodiments, the lung cancer is refractory. In some embodiments, the lung cancer is refractory to treatment with carboplatin. In some embodiments, the non-small cell lung cancer is refractory. In some embodiments, the non-small cell lung cancer is refractory to treatment with carboplatin. In some embodiments, the lung cancer is refractory to treatment with erlotinib, gefitinib, osimertinib, or crizotinib. In some embodiments, the lung cancer is refractory to treatment with pemetrexed, docetaxol, or pembroluzimab. In some embodiments, the lung cancer is refractory to erlotinib, gefitinib, osimertinib, crizotinib, pemetrexed, docetaxol, or pembroluzimab. In some embodiments, the non-small cell lung cancer is refractory to treatment with erlotinib, gefitinib, osimertinib, or crizotinib. In some embodiments, the non-small cell lung cancer is refractory to treatment with pemetrexed, docetaxol, or pembroluzimab. In some embodiments, the non-small cell lung cancer is refractory to erlotinib, gefitinib, osimertinib, crizotinib, pemetrexed, docetaxol, or pembroluzimab. In some embodiments, the lung cancer is refractory to gemcitabine, bortexomib, trastuzumab, vinorelbine, doxorubicin, irinotecan, temsirolimus, sunitinib, nivolumab, or bevacizumab. In some embodiments, the lung cancer is refractory to carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the non-small cell lung cancer is refractory to gemcitabine, bortexomib, trastuzumab, vinorelbine, doxorubicin, irinotecan, temsirolimus, sunitinib, nivolumab, or bevacizumab. In some embodiments, the non-small cell lung cancer is refractory to carboplatin/gemcitabine, carboplatin/paclitaxel/cetuximua, cisplatin/pemetrexed, cisplatin/docetaxel, cisplatin/docetaxel/bevacizumab, everolimus/nab-paclitaxel, or tremelimumab/durvalumab. In some embodiments, the subject with lung cancer comprises a mutation in KRAS, PTEN, TSC1, TSC2, PIk3CA, P53, STK11 (a.k.a. LKB1), KEAP1, NRF2, ALK4, GNAS or EGFR.

In some embodiments, the cancer is breast cancer. In some embodiments, the breast cancer comprises a tumor. In some embodiments, the breast cancer is characterized by abnormal autophagy. In some embodiments, the breast cancer is refractory. In some embodiments, the breast cancer is refractory to anastrozole, exemestane, letrozole, or tamoxifen. In some embodiments, the breast cancer is refractory to a poly ADP ribose polymerase (PARP) inhibitor. In some embodiments, the breast cancer is refractory to anastrozole, exemestane, letrozole, tamoxifen, or a PARP inhibitor. In some embodiments, the PARP inhibitor is olaparib, rucaparib, niraparib, or talazoparib. In some embodiments, the breast cancer is refractory to olaparib, rucaparib, niraparib, or talazoparib. In some embodiments, the breast cancer is triple negative breast cancer.

In some embodiments, the cancer is pancreatic cancer. In some embodiments, the pancreatic cancer comprises a tumor. In some embodiments, the pancreatic cancer is characterized by abnormal autophagy. In some embodiments, the pancreatic cancer is refractory. In some embodiments, the pancreatic cancer is refractory to FOLFIRINOX (5-fluorouracil, leucovorin, irinotecan, and oxaliplatin), gemcitabine, or gemcitabine/abraxane. In some embodiments, the pancreatic cancer is refractory. In some embodiments, the pancreatic cancer is refractory to FOLFIRINOX (5-fluorouracil, leucovorin, irinotecan, and oxaliplatin), gemcitabine, gemcitabine/abraxane, everolimus, erlotinib, or sunitinib. In some embodiments, the pancreatic cancer is refractory to gemcitabine. In some embodiments, the pancreatic cancer is refractory to capeditabine, leucovorin, nab-paclitaxel, nanoliposomal irinotecan, gemcitabine/nab-paclitaxel, pembrolizumab, or cisplatin. In some embodiments, the pancreatic cancer is pancreatic ductal adenocarcinoma (PDAC). In some embodiments, the subject with pancreatic cancer comprises a mutation in at least one of SMAD4, p16/CDKM2A, or BRCA2.

In some embodiments, the disease or disorder treated with a ULK inhibitor as a monotherapy is lymphangiomyomatosis. In some embodiments, the disease or disorder treated with a ULK inhibitor as a monotherapy is tuberous sclerosis complex.

In some embodiments, administering a ULK inhibitor slows progression of the disease or disorder. In some embodiments, administering a ULK inhibitor slows progression of the disease or disorder by at least 10%, at least 20%, at least 30%, at least 40%, at least 50%, at least 60%, at least 70%, at least 80%, at least 90%, or at least 95%. In some embodiments, progression is measured by tumor growth. In some embodiments, administering a ULK inhibitor arrests cancer cell growth. In some embodiments, administering a ULK inhibitor reduces tumor volume. In some instances, the ULK inhibitor inhibits ULK1. In some instances, the ULK inhibitor is a ULK1 specific inhibitor. In some instances, the ULK inhibitor inhibits both ULK1 and ULK2.

In some embodiments, the method of treatment comprises decreasing phosphorylation of ATG13 in the subject. In some embodiments, the method comprises degrading ATG13 in diseased tissue of the subject.

In some embodiments, the additional therapeutic agent is carboplatin. In some embodiments, the additional therapeutic agent is a carboplatin analog. In some embodiments, the carboplatin analog is cisplatin or dicycloplatin.

In some embodiments, the additional therapeutic agent is an MEK inhibitor. In some embodiments, the additional therapeutic agent is trametinib. In some embodiments, the MEK inhibitor is trametinib, cobimetinib, binimetinib, or selumetinib. In some embodiments, the additional therapeutic agent is G12C inhibitor.

In some embodiments, the additional therapeutic agent is gemcitabine. In some embodiments, the additional therapeutic agent is a nucleoside analog.

In some embodiments, the additional therapeutic agent is a poly ADP ribose polymerase (PARP) inhibitor. In some embodiments, the PARP inhibitor is olaparib, rucaparib, niraparib, or talazoparib. In some embodiments, the additional therapeutic agent is olaparib, rucaparib, niraparib, or talazoparib.

In some embodiments, the additional therapeutic agent is erlotinib, gefitinib, osimertinib, or crizotinib. In some embodiments, the additional therapeutic agent is anastrozole, exemestane, letrozole, or tamoxifen. In some embodiments, the additional therapeutic agent is gemcitabine, everolimus, erlotinib, or sunitinib. In some embodiments, the additional therapeutic agent is erlotinib, gefitinib, osimertinib, crizotinib, pemetrexed, docetaxol, or pembroluzimab.

In some embodiments, the subject is treated with the additional therapeutic agent prior to treatment with the ULK inhibitor. In some embodiments, treatment with the additional therapeutic agent is ceased prior to administration of the ULK inhibitor. In some embodiments, treatment with the additional therapeutic agent produces a cytostatic response in diseased tissue.

In some embodiments, the ULK inhibitor and the additional therapeutic agent are administered concomitantly. In some embodiments, the ULK inhibitor and the additional therapeutic agent are administered together at the start of treatment.

Aspects of the disclosure include use of a ULK inhibitor, as described herein, in the preparation of a medicament for the treatment of a disease or disorder characterized by abnormal autophagy, abnormal ULK1 activity, abnormal ULK2 activity, or any combination thereof. Aspects of the disclosure include ULK inhibitors, as described herein, for use in the treatment of a disease or disorder characterized by abnormal autophagy, abnormal ULK1 activity, abnormal ULK2 activity, or any combination thereof.

Aspects of the disclosure include kits comprising the active agents (e.g., ULK inhibitors) and formulations thereof, of the invention and instructions for use. A kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

### Gene Profile

Effectiveness of treatment of a disorder mediated by ULK using ULK inhibitors in either monotherapy or combination therapy can vary based on a status of a disease or disorder. In some embodiments, the disorder is a cancer. In some embodiments, the status of the disorder comprises a mutation status of a tissue affected by the disorder (e.g., types of mutations in one or more genes, or in specific genes), and/or a gene expression profile of the tissue affected by the disorder. Thus, disclosed herein are methods of treating a disorder mediated by ULK in a subject in need thereof by administering to the subject a therapeutically effective amount of a ULK inhibitor, wherein the tissue in the subject has a distinct expression of at least one biomarker gene. Also disclosed herein are methods of predicting a likelihood of success of treating a disorder mediated by ULK with a ULK inhibitor in a subject in need thereof by obtaining a gene expression profile of a plurality of genes from a tissue of the subject and predicting the likelihood of success of a ULK inhibitor treatment based on the gene profile. Also disclosed herein are methods of selecting a subject for a ULK inhibitor treatment against a cancer in the patient by obtaining a gene expression profile of a plurality of genes from a tissue of the subject and selecting the subject for the ULK inhibitor treatment based on the gene profile. In some embodiments, the tissue is a cancer tissue.

Table 1 shows exemplary genes that are highly expressed in a cancer tissue that are very responsive to the ULK inhibitor treatment. Alternatively, Table 1 shows exemplary genes that are expressed lower in a cancer tissue that are not responsive or less responsive to the ULK inhibitor treatment. Thus, in some embodiments, the likelihood of success of a ULK inhibitor treatment is predicted to be high when a gene expression level of the at least one, at least two, at least three, at least four, at least five, at least six, a least seven, at least eight, at least nine, at least ten gene in Table 1 is above a predetermined threshold in a cancer or cancer cell treated with an ULK inhibitor. Alternatively and/or additionally, the subject is selected for the ULK inhibitor treatment when the gene expression level of the at least one, at least two, at least three, at least four, at least five, at least six, a least seven, at least eight, at least nine, at least ten genes in Table 1 is above a predetermined threshold in a cancer or cancer cell treated with an ULK inhibitor.

Table 2 shows exemplary genes that are expressed lower in a cancer tissue that are very responsive to the ULK inhibitor treatment. Alternatively, Table 2 shows exemplary genes that are expressed highly in a cancer tissue that are not responsive or less responsive to the ULK inhibitor treatment. Thus, in some embodiments, the likelihood of success of a ULK inhibitor treatment is predicted high when gene expression level of the at least one, at least two, at least three, at least four, at least five, at least six, a least seven, at least eight, at least nine, at least ten genes in Table 1 is below a predetermined threshold in a cancer or cancer cell treated with an ULK inhibitor. Alternatively and/or additionally, the subject is selected for the ULK inhibitor treatment when gene expression level of the at least one, at least two, at least three, at least four, at least five, at least six, a least seven, at least eight, at least nine, at least ten genes in Table 1 is below a predetermined threshold in a cancer or cancer cell treated with an ULK inhibitor.

TABLE 1

| Gene Name | UniProt Number |
|---|---|
| FUZ | Q9BT04 |
| EDN1 | P05305 |
| DUSP8 | Q13202 |
| HGD | Q93099 |
| SLC51A | Q86UW1 |
| SYT17 | Q9BSW7 |
| SEL1L3 | Q68CR1 |
| RASSF7 | Q02833 |
| PCBD2 | Q9H0N5 |
| NUDT22 | Q9BRQ3 |
| CAMLG | P49069 |
| CASP7 | P55210 |
| HSD17B14 | Q9BPX1 |
| LTA4H | P09960 |
| SLC25A37 | Q9NYZ2 |
| NAMPT | P43490 |
| C15orf48 | Q9C002 |
| STK32A | Q8WU08 |
| ST3GAL1 | Q11201 |
| VMP1 | Q96GC9 |
| EPHX3 | Q9H6B9 |
| LPCAT1 | Q8NF37 |
| SLC22A31 | A6NKX4 |
| FAM177A1 | Q8N128 |
| CARD16 | Q5EG05 |
| SLC34A2 | O95436 |
| KCNQ3 | O43525 |
| MSRB1 | Q9NZV6 |
| TMC5 | Q6UXY8 |
| ABCC3 | O15438 |
| RPL19 | P84098 |
| MAL2 | Q969L2 |
| ZNF341 | Q9BYN7 |
| PDZK1IP1 | Q13113 |
| RHBDL1 | O75783 |
| KCNMB4 | Q86W47 |
| ESRRA | P11474 |
| FAM89B | Q8N5H3 |
| FUT2 | Q10981 |
| FAM174A | Q8TBP5 |
| SLC22A18AS | Q8N1D0 |
| SLC8B1 | Q6J4K2 |
| NOL3 | O60936 |
| CCDC88B | A6NC98 |
| MISP | Q8IVT2 |
| CAPN8 | A6NHC0 |
| CSTB | P04080 |
| SAA2 | P0DJI9 |
| C3orf55 | A1A4F0 |
| ORMDL2 | Q53FV1 |
| SIPA1 | Q96FS4 |
| SAA1 | P0DJI8 |
| RP4-583P15.14 | Q9H400 |
| (LIME) | |
| UNC93B1 | Q9H1C4 |
| DCPS | Q96C86 |
| MMP15 | P51511 |
| PKP3 | Q9Y446 |
| NR1H3 | Q13133 |
| HPN | P05981 |
| ST5 | P78524 |
| SIGIRR | Q6IA17 |
| ERBB3 | P21860 |
| CRB3 | Q9BUF7 |
| MARVELD3 | Q96A59 |
| ABHD11 | Q8NFV4 |
| CLDN7 | O95471 |
| RBPMS | Q93062 |
| BSPRY | Q5W0U4 |
| TP53I13 | Q8NBR0 |
| EPCAM | P16422 |
| SERF1B | O75920 |
| STEAP4 | Q687X5 |
| RPL27 | P61353 |
| RPL23 | P62829 |
| CDH1 | CDH1 |
| LENG9 | Q96B70 |
| CEACAM19 | Q7Z692 |
| LLGL2 | Q6P1M3 |
| REEP6 | Q96HR9 |
| METRN | Q9UJH8 |
| CORO1A | P31146 |
| CWC15 | Q9P013 |
| SMUG1 | Q53HV7 |
| ANKRD13D | Q6ZTN6 |
| EVPL | Q92817 |
| MRPL53 | Q96EL3 |
| TBX6 | O95947 |
| SMIM22 | K7EJ46 |
| TMEM134 | Q9H6X4 |
| FKSG61 | Q9BZ62 |
| SPATA20 | Q8TB22 |
| ACP5 | P13686 |
| CPLX1 | O14810 |
| NSUN5 | Q96P11 |
| KRTCAP3 | Q53RY4 |
| RPP25 | Q9BUL9 |
| C10orf35 | Q96D05 |
| COL18A1 | P39060 |
| GDPD3 | Q7L5L3 |
| P2RX4 | Q99571 |
| TMEM179B | Q7Z7N9 |
| TLCD1 | Q96CP7 |
| KRT10 | P13645 |
| SLC50A1 | Q9BRV3 |
| FSCN2 | O14926 |
| EME2 | A4GXA9 |
| EIF4EBP3 | O60516 |
| TSPAN15 | O95858 |
| NOS3 | P29474 |
| MCU | Q8NE86 |
| NME1-NME2 | Q32Q12 |
| ASCC2 | Q9H1I8 |
| IFITM2 | Q01629 |
| HKDC1 | Q2TB90 |
| ATP5H | O75947 |
| FAM21D | Q5SRD0 |
| RPL35A | P18077 |
| RGS17 | Q9UGC6 |
| GCHFR | P30047 |
| TK1 | P04183 |
| MRPS34 | P82930 |
| TOP1 | P11387 |
| SAMD4A | Q9UPU9 |
| MRGBP | Q9NV56 |
| RAB27A | P51159 |
| ADK | P55263 |
| SDR16C5 | Q8N3Y7 |
| DDX27 | Q96GQ7 |
| GPX4 | P36969 |
| TMEM50A | O95807 |
| MARVELD1 | Q9BSK0 |
| PUSL1 | Q8N0Z8 |
| XAGE1C | Q9HD64 |
| XAGE1A | Q9HD64 |
| XAGE1E | Q9HD64 |
| XAGE1D | Q9HD64 |
| XAGE1B | Q9HD64 |
| S100A6 | P06703 |
| H2AFJ | Q9BTM1 |
| SNRPD3 | P62318 |
| NUP85 | Q9BW27 |
| NOL12 | Q9UGY1 |
| NT5C | Q8TCD5 |
| MRPL27 | Q9P0M9 |
| CBLC | Q9ULV8 |
| GFAP | P14136 |
| CBY1 | Q9Y3M2 |
| DPM2 | O94777 |
| TMEM150A | Q86TG1 |
| ABHD14B | Q96IU4 |
| MPST | P25325 |
| SCO2 | O43819 |
| KIAA0930 | Q6ICG6 |
| STK32C | Q86UX6 |
| PLEKHJ1 | Q9NW61 |
| PPA1 | Q15181 |
| IFITM3 | Q01628 |
| PTRH2 | Q9Y3E5 |
| UQCR10 | Q9UDW1 |
| NME1 | P15531 |
| AMN | Q9BXJ7 |
| ABCC6 | O95255 |
| NARS2 | Q96159 |
| SMPDL3B | Q92485 |
| CHMP2A | O43633 |
| SFTA2 | Q6UW10 |

TABLE 2

| Gene Name | UniProt Number |
|---|---|
| SASH1 | O94885 |
| USP5 | P45974 |
| ZFYVE9 | O95405 |
| TMX4 | Q9H1E5 |
| APH1B | Q8WW43 |
| KDM5A | P29375 |
| CLSPN | Q9HAW4 |
| SENP1 | Q9P0U3 |
| SMYD4 | Q8IYR2 |
| XXYLT1 | Q8NBI6 |
| ZNF451 | Q9Y4E5 |
| ARHGEF37 | A1IGU5 |
| METTL7A | Q9H8H3 |
| CDON | Q4KMG0 |
| RPA1 | P27694 |
| MRPL19 | P49406 |
| RAB23 | Q9ULC3 |
| PHLDB2 | Q86SQ0 |
| HNRNPLL | Q8WVV9 |
| VAV2 | P52735 |
| PHF10 | Q8WUB8 |
| STX17 | P56962 |
| MSH6 | P52701 |
| MANEA | Q5SRI9 |
| GNGT1 | P63211 |
| PLXNA2 | O75051 |
| C6orf106 | Q9H6K1 |
| MYCBP2 | O75592 |
| FEM1B | Q9UK73 |
| ASXL2 | Q76L83 |
| HIF1AN | Q9NWT6 |
| ATRN | O75882 |
| SOCS5 | O75159 |
| RXRB | P28702 |
| ZBTB9 | Q96C00 |
| MCOLN3 | Q8TDD5 |
| ULBP3 | Q9BZM4 |
| BOC | Q9BWV1 |
| TSPAN9 | O75954 |
| SMIM10 | Q96HG1 |
| ZNF595 | Q8IYB9 |
| RAB36 | O95755 |
| SEMA3F | Q13275 |
| SORBS2 | O94875 |
| PDSS2 | Q86YH6 |
| NSD1 | Q96L73 |
| STC1 | P52823 |
| MAML1 | Q92585 |
| CANX | P27824 |
| FAM229B | Q4G0N7 |
| FAM46A | Q96IP4 |
| MAN1A2 | O60476 |
| HSP90AA1 | P07900 |
| FIG4 | Q92562 |
| CHN1 | P15882 |
| WARS | P23381 |
| GLIPR2 | Q9H4G4 |
| TNFRSF19 | Q9NS68 |
| MGP | P08493 |
| RECK | O95980 |
| ST8SIA1 | Q92185 |
| FBN2 | P35556 |
| TTLL7 | Q6ZT98 |
| CTTNBP2 | Q8WZ74 |
| MYB | P10242 |
| GSG2 | Q8TF76 |
| MCM3 | P25205 |
| SIRPA | P78324 |
| KIAA1549 | Q9HCM3 |
| AMD1 | P17707 |
| ITIH4 | Q14624 |
| NPTXR | O95502 |
| PTPRN2 | Q92932 |
| PCDH18 | Q9HCL0 |
| ANXA6 | P08133 |
| CAMK2N2 | Q96S95 |
| SCARA3 | Q6AZY7 |
| TGFBR3 | Q03167 |
| EIF5 | P55010 |
| HBS1L | Q9Y450 |
| KIAA0232 | Q92628 |
| SSPN | Q14714 |
| BTBD3 | Q9Y2F9 |
| PEX7 | O00628 |
| PPT2 | Q9UMR5 |
| DPF1 | Q92782 |
| RC3H2 | Q9HBD1 |
| TIAM2 | Q8IVF5 |
| CPA4 | Q9UI42 |
| NUSAP1 | Q9BXS6 |
| UBOX5 | O94941 |
| TULP4 | Q9NRJ4 |
| ARID1B | Q8NFD5 |
| PTPRK | Q15262 |
| PRKACB | P22694 |
| HACE1 | Q8IYU2 |
| BAG5 | Q9UL15 |
| LATS1 | O95835 |
| ERMP1 | Q7Z2K6 |
| SCAF8 | Q9UPN6 |
| ATP1A1 | P05023 |
| MED23 | Q9ULK4 |
| TBP | P20226 |
| RBP1 | P09455 |
| EPM2A | O95278 |
| TMEM181 | Q9P2C4 |
| CHN2 | P52757 |
| PPP1R3C | Q9UQK1 |
| PRKD3 | O94806 |
| MCM8 | Q9UJA3 |
| FAM169A | Q9Y6X4 |
| SH3BP4 | Q9P0V3 |
| LMO4 | P61968 |
| WDR54 | Q9H977 |
| MAP3K7 | O43318 |
| NUP43 | Q8NFH3 |
| ZNF326 | Q5BKZ1 |
| MTO1 | Q9Y2Z2 |
| MMS22L | Q6ZRQ5 |
| GNMT | Q14749 |
| MAN2A2 | P49641 |
| MEGF8 | Q7Z7M0 |
| SPRYD3 | Q8NCJ5 |
| RFC1 | P35251 |
| WDR81 | Q562E7 |
| CSRP2BP | Q9H8E8 |
| RAB8B | Q92930 |
| GNL1 | P36915 |
| MCOLN2 | Q8IZK6 |
| MRAS | O14807 |
| SF3A3 | Q12874 |
| SYS1-DBNDD2 | H3BUS1 |
| TBPL1 | P62380 |
| CHAF1A | Q13111 |
| SAE1 | Q9UBE0 |
| PARD3B | Q8TEW8 |
| IDS | P22304 |
| AXIN2 | Q9Y2T1 |
| TLN2 | Q9Y4G6 |
| DCLK2 | Q8N568 |
| ENPP1 | P22413 |
| WDR25 | Q64LD2 |
| UFL1 | O94874 |
| SLC26A2 | P50443 |
| NHSL1 | Q5SYE7 |
| PDE7A | Q13946 |
| IGFBP5 | P24593 |
| TPM1 | P09493 |
| ZZEF1 | O43149 |
| ZNF17 | P17021 |
| USP49 | Q70CQ1 |
| SOGA1 | O94964 |
| DISC1 | Q9NRI5 |
| APOLD1 | Q96LR9 |
| CDK19 | Q9BWU1 |
| ZSCAN12 | O43309 |
| SLC25A30 | Q5SVS4 |
| KIAA0753 | Q2KHM9 |
| TDP2 | O95551 |
| FZD1 | Q9UP38 |
| PPIL6 | Q8IXY8 |
| NFATC3 | Q12968 |
| RECQL | P46063 |
| TCF12 | Q99081 |
| TSR1 | Q2NL82 |
| STK38L | Q9Y2H1 |
| DPP8 | Q6V1X1 |
| FANCE | Q9HB96 |
| NOL10 | Q9BSC4 |
| COL5A1 | P20908 |
| RRM2 | P31350 |
| DICER1 | Q9UPY3 |
| PVRL1 | Q15223 |
| ARHGAP23 | Q9P227 |
| MMP2 | P08253 |
| CXorf57 | Q6NSI4 |
| SH3RF3 | Q8TEJ3 |
| NCAPD2 | Q15021 |
| TRAM2 | Q15035 |
| ZNF229 | Q9UJW7 |
| ZMIZ1 | Q9ULJ6 |
| PTPRA | P18433 |
| STXBP1 | P61764 |
| APBB2 | Q92870 |
| NXN | Q6DKJ4 |
| LTBP1 | Q14766 |
| EXOC2 | Q96KP1 |
| ABCF1 | Q8NE71 |
| CMTR1 | Q8N1G2 |
| ABR | Q12979 |
| TAF11 | Q15544 |
| C2orf44 | Q9H6R7 |
| LRRC28 | Q86X40 |
| SRRM1 | Q8IYB3 |
| BACH1 | O14867 |
| AAGAB | Q6PD74 |
| ZNF697 | Q5TEC3 |
| MAPK10 | P53779 |
| DNAJC27 | Q9NZQ0 |
| PSPH | P78330 |
| HS2ST1 | Q7LGA3 |
| LANCL1 | O43813 |
| IREB2 | P48200 |
| YOD1 | Q5VVQ6 |
| GABPA | Q06546 |
| ZNF770 | Q6IQ21 |
| MPDZ | O75970 |
| EXTL2 | Q9UBQ6 |
| ZNF644 | Q9H582 |
| TMEM45A | Q9NWC5 |
| MORC3 | Q14149 |
| TRMT61B | Q9BVS5 |
| C20orf194 | QSTEA3 |
| GPR125 | Q8IWK6 |
| CD82 | P27701 |
| WDR35 | Q9P2L0 |
| MCC | P23508 |
| MED21 | Q13503 |
| ARL4A | P40617 |
| HMG20A | Q9NP66 |
| KIAA1586 | Q9HCI6 |
| ZKSCAN8 | Q15776 |
| RPS6KA5 | O75582 |
| RNF168 | Q8IYW5 |
| CNOT6 | Q9ULM6 |
| ZNF362 | Q5T0B9 |
| PUM2 | Q8TB72 |
| ARHGAP28 | Q9P2N2 |
| BCKDHB | P21953 |
| POLR2A | P24928 |
| AGL | P35573 |
| NIPAL1 | Q6NVV3 |
| EDARADD | Q8WWZ3 |
| Col4A4 | P53420 |
| BMP7 | P18075 |
| GNE | Q9Y223 |
| Parp11 | Q9NR21 |
| ACO10441.1 | Q5W111 |
| (SPRYD7) | |
| ERCC6L2 | Q5T890 |
| PPM1B | O75688 |
| ZBTB33 | Q86T24 |
| ZNF562 | Q6V9R5 |
| ZNF845 | Q96IR2 |
| DENND6A | Q8IWF6 |
| TOX4 | O94842 |
| TRIP11 | Q15643 |
| EXOC5 | O00471 |
| C21orf91 | Q9NYK6 |
| UBE2G1 | P62253 |
| HSPA13 | P48723 |
| LRP1 | Q07954 |
| CTBS | Q01459 |
| KLHL13 | Q9P2N7 |
| FOXJ2 | Q9P0K8 |
| GPC4 | O75487 |
| DPYSL3 | Q14195 |
| BIVM-ERCC5 | R4GMW8 |
| VTA1 | Q9NP79 |
| DEPDC1 | Q5TB30 |
| WTAP | Q15007 |
| SYNCRIP | O60506 |
| FBXO5 | Q9UKT4 |
| SSX2IP | Q9Y2D8 |
| STIL | Q15468 |
| ALDH9A1 | P49189 |
| NT5DC1 | Q5TFE4 |

In some embodiments, the genes selected for a gene profile comprise FUZ, EDN1, DUSP8, HGD, SLC51A, SYT17, SEL1L3, RASSF7, PCBD2, NUDT22, CAMLG, CASP7, HSD17B14, LTA4H, SLC25A37, NAMPT, C15orf48, STK32A, or ST3GAL1. Alternatively and/or additionally, the genes selected for a gene profile comprise SASH1, USP5, ZFYVE0, TMX4, APH1B, KDM5A, CLSPN, SENP1, SMYD4, XXYLT1, ZNF451, ARHGEF37, METTL7A, CDON, RPA1, MRPL19, RAB23, PHLDB2, or HNRNPLL.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms include quantitative, qualitative or quantitative and qualitative determinations. Assessing may be relative or absolute. In some embodiments, “detecting the presence of” includes determining the amount of something present in addition to determining whether it is present or absent depending on the context.

The terms “subject,” “individual,” or “patient” are often used interchangeably herein. A “subject” is a biological entity containing expressed genetic materials. In some embodiments, the biological entity is a plant, animal, or microorganism, including, for example, bacteria, viruses, fungi, and protozoa. In some embodiments, the subject comprises tissues, cells and their progeny of a biological entity obtained in vivo or cultured in vitro. In some embodiments, the subject is a mammal. In some embodiments, the mammal is a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In some embodiments, in vitro assays encompass cell-based assays in which living or dead cells are employed. In some embodiments, in vitro assays also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. In some embodiments, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, “monotherapy” means a therapy that uses a single drug to treat a disease or condition. The single drug may be used in conjunction with various inactive ingredients, such as those used in a formulation to improve pharmaceutical properties. This is compared to the term “combination therapy,” wherein two or more therapeutic agents are administered concomitantly.

The section headings used herein are for organizational purposes only and are not to be construed as limiting the subject matter described.

### EXAMPLES

The following examples are included for illustrative purposes only and are not intended to limit the scope of the invention.

### Example 1: Biomarker Identification for Predicting Response to ULK1 Inhibitors

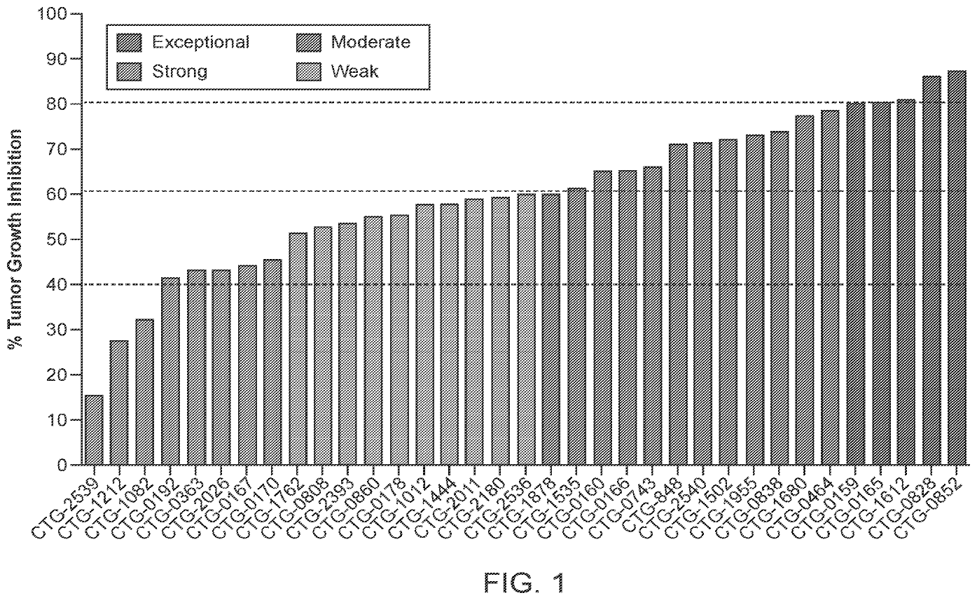

35 patient derived xenograft models were injected into the flanks of nude mice and treated with Ulk1 inhibitors (40mg/kg QD) or vehicle control once tumors reached 150-300 mm³ as measured by calipers twice a week (3 mice per group). Mice were treated until tumors reached endpoint of 1500 mm³ and percent tumor growth inhibition was calculated using the formula [100−(final MVT treated/final MVT control*100)]. These 35 patients' tissues are associated with patients' outcome. FIG. 1 shows tumor growth inhibition graphs of 35 patients derived xenograft models. Models with less than 40% response were categorized as “non-responders”, 40-59% as “weak” responders, 60-79% as “strong responders” and 80%-100% as “exceptional responders”.

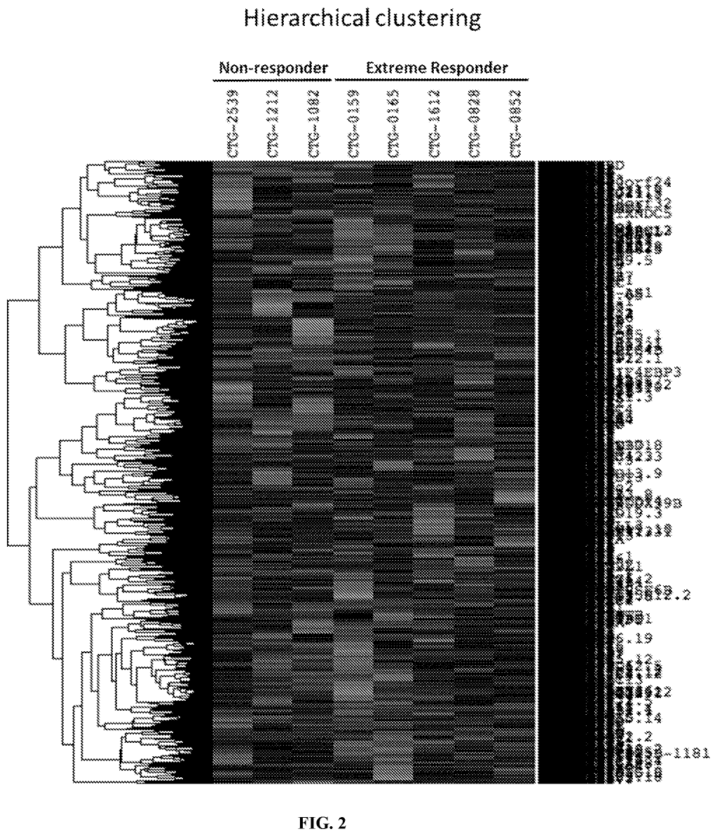

To identify gene signatures for predicting patient response to ULK1 inhibitors, RNAseq data from the 3 non-responder and 5 extreme responder patient derived xenograft samples (pre-treatment) were analyzed to identify genes that were differentially expressed across these two groups of tumors. Log2(RPKM+1) data was filtered, normalized, and clustered using CLUSTER software as follows: gene were required to have at least 2 observations with abs(val)>=0.7 to filter out minimally-expressed genes and a 1.5 cutoff was used to generate a list of 10,709 genes that had at least a 3 fold change between the highest and lowest-expressing models. These genes were then centered around the mean and normalized to allow for hierarchical clustering using average linkages. Heatmaps were generated using Java TreeView software and two types of clusters were identified: 1) genes with high expression in all of the extreme responders compared to non-responders (UP) and 2) genes with low expression all of the extreme responders compared to non-responders (DOWN). From these clusters, we identified a list of 167 UP and 258 DOWN genes that comprise a potential signature of response. FIG. 2 shows a heatmap of the hierarchical clustering of genes that are differentially expressed in non-responders and extreme responders.

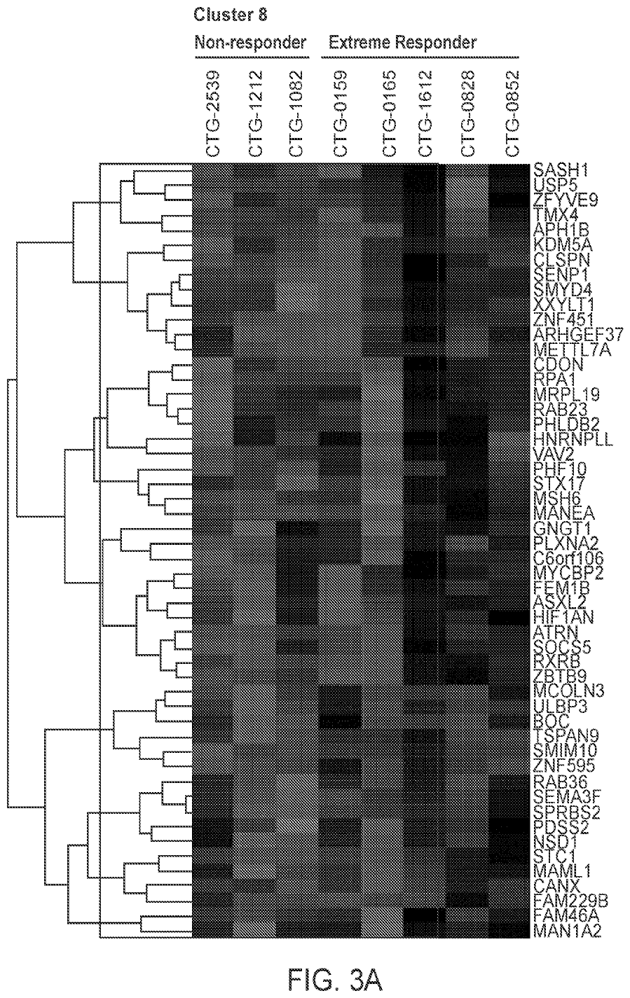

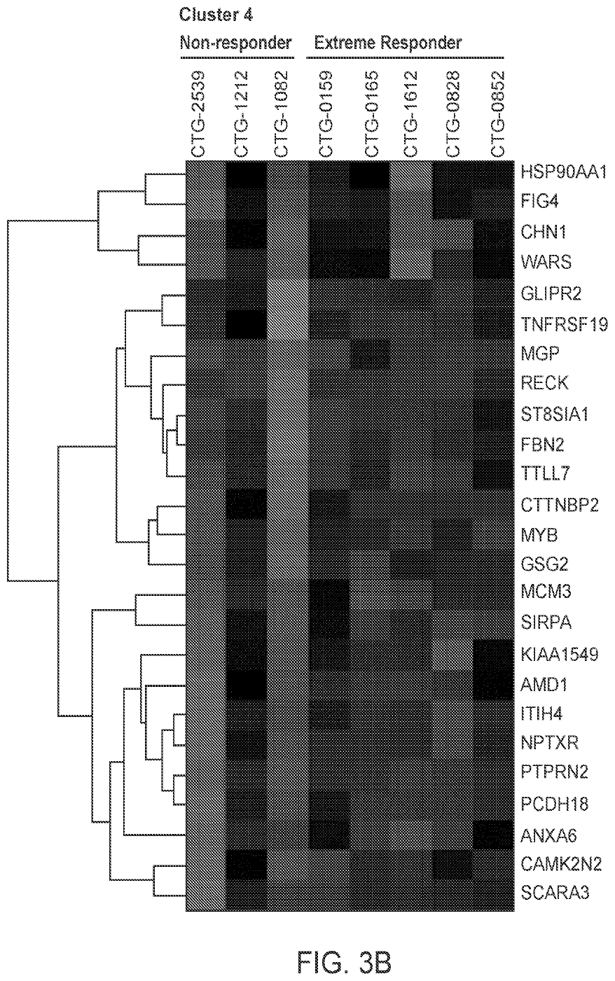

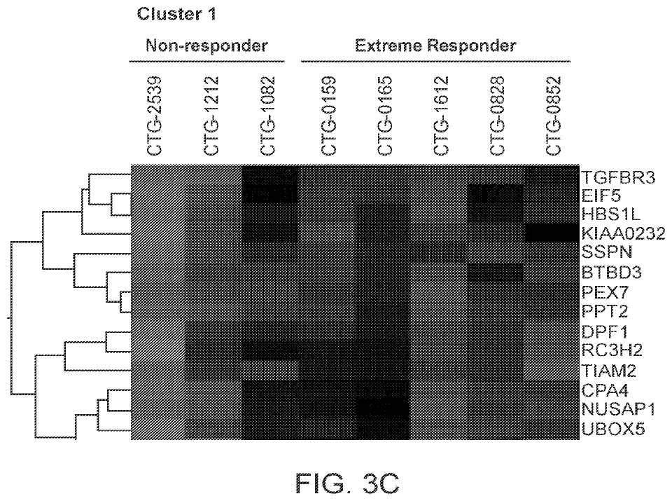

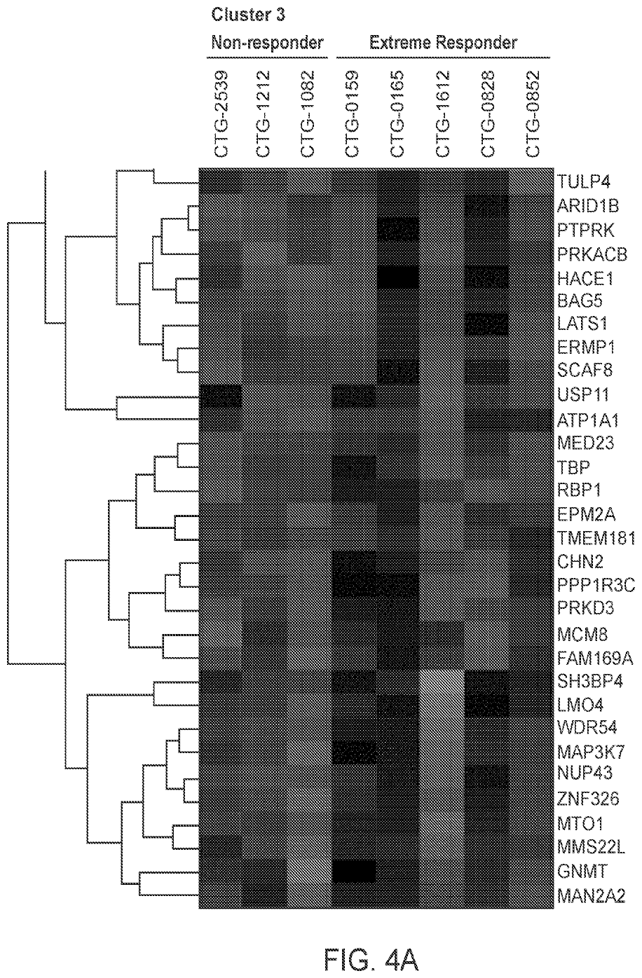

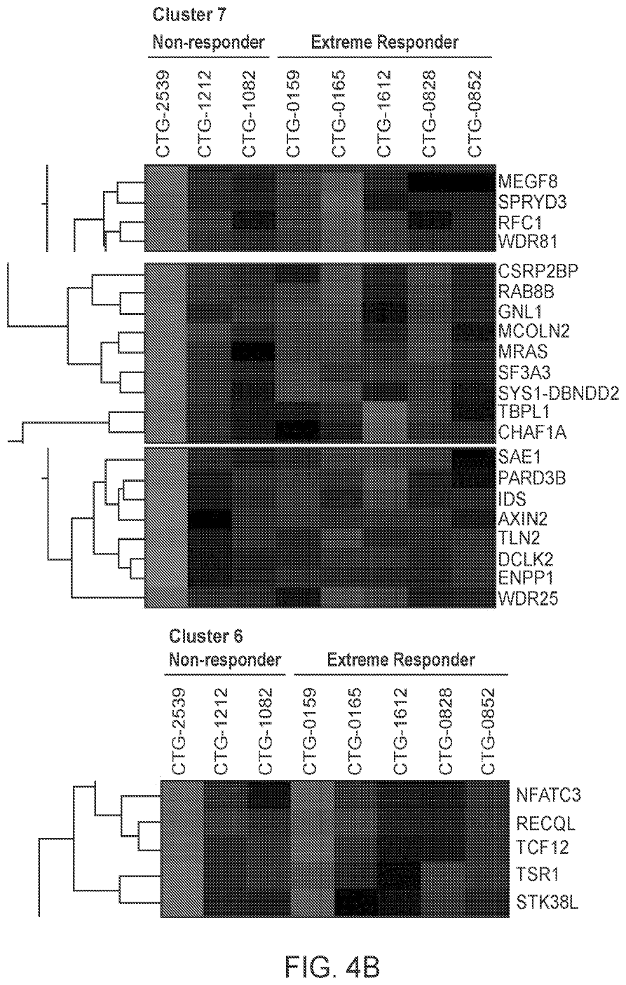

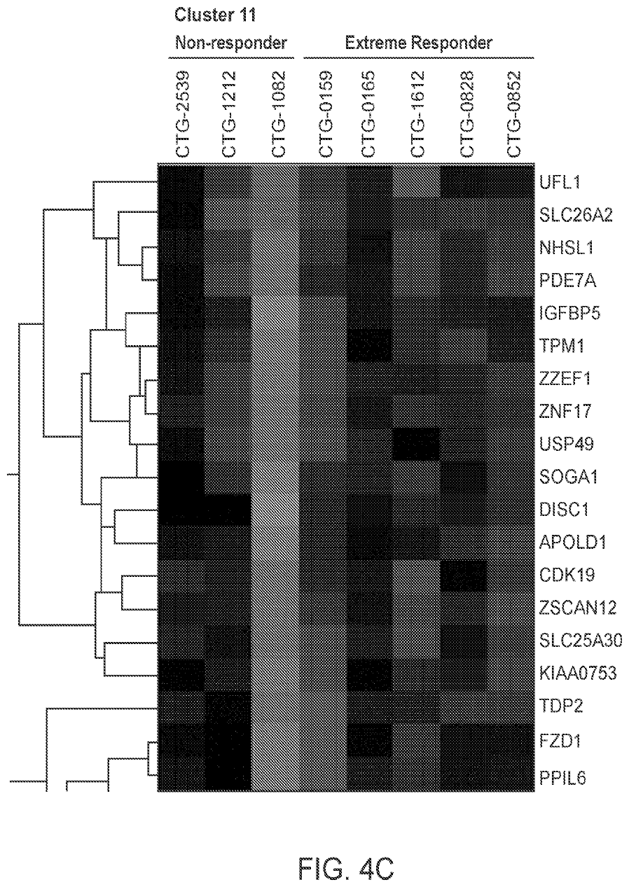

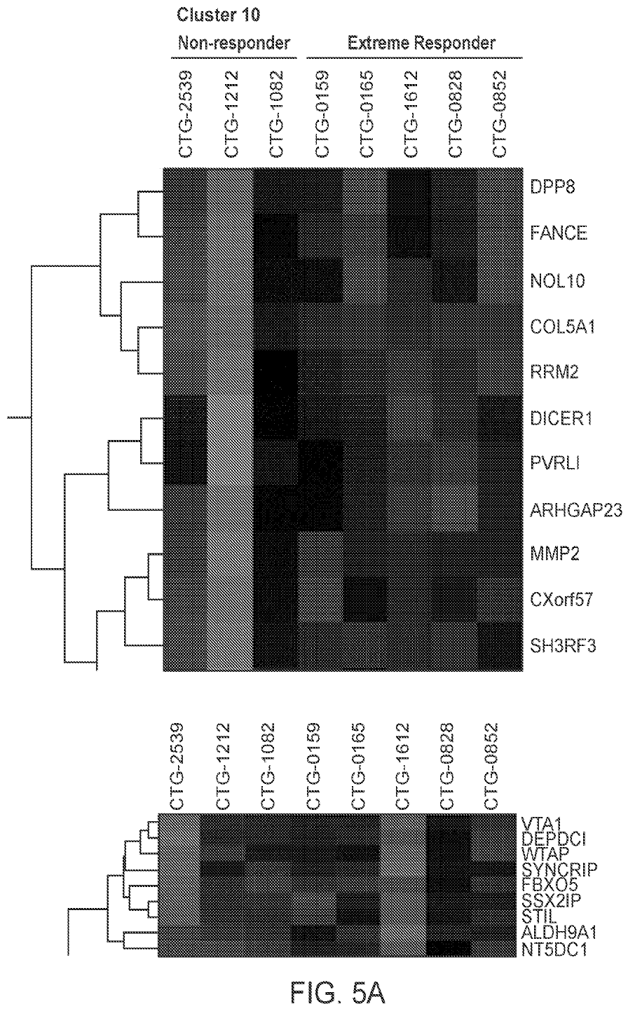

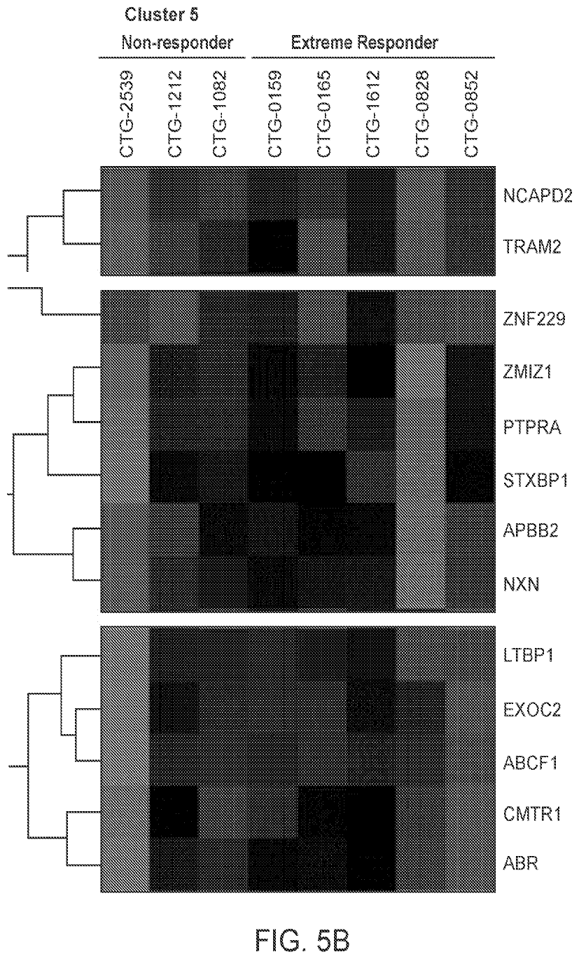

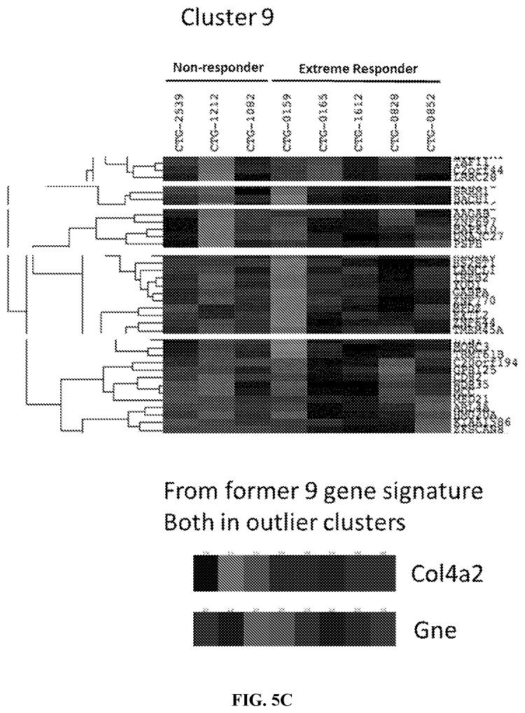

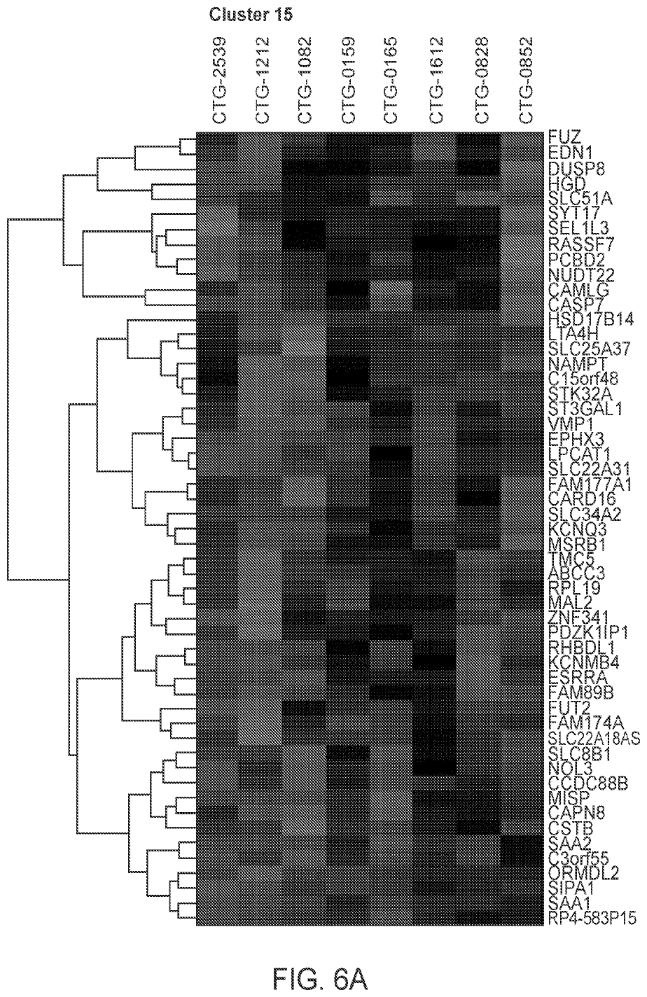

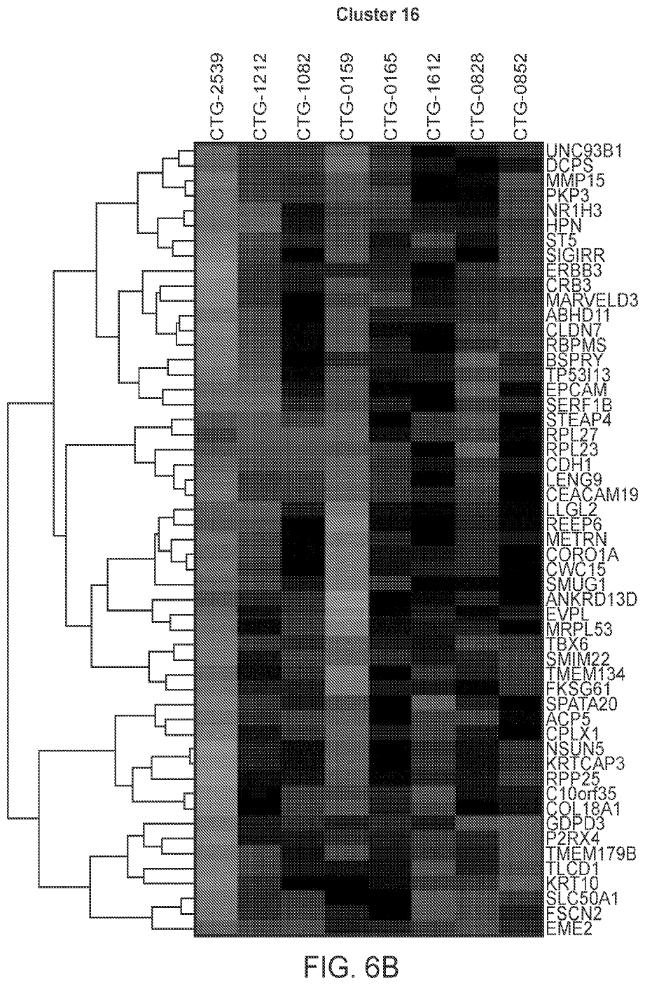

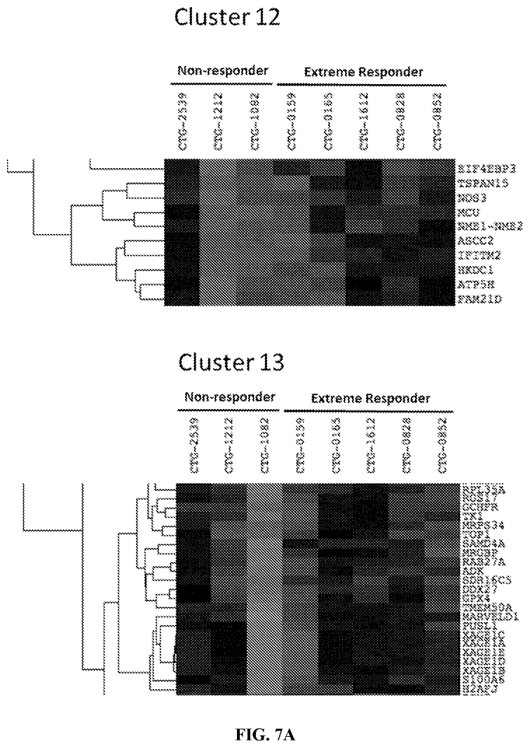

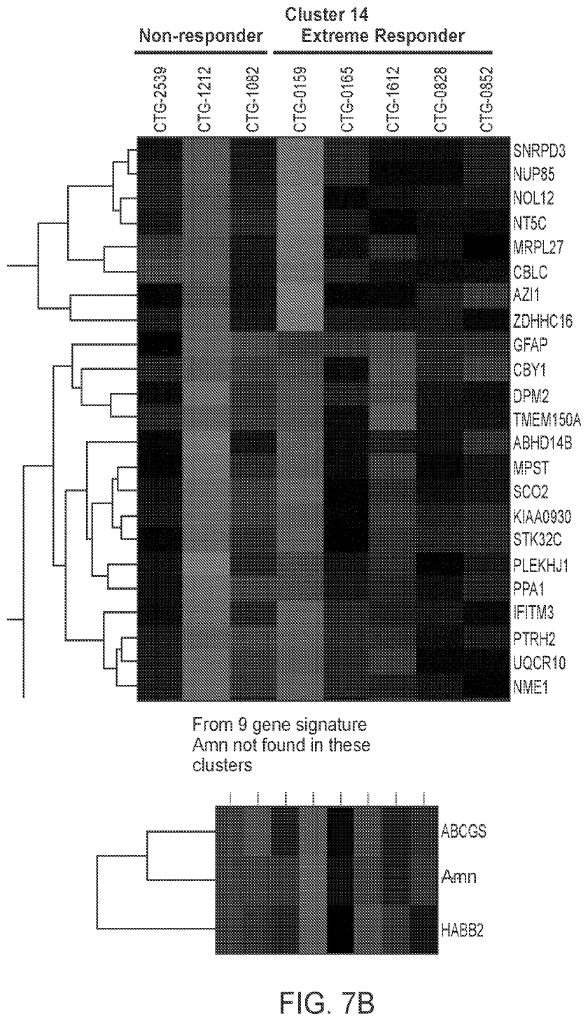

FIGS. 3A-C, FIGS. 4A-C, FIGS. 5A-C show heat maps of various gene clusters (266 genes) that show lower expression levels in extreme responders. FIGS. 6A-B, FIGS. 7A-B show heat maps of variousgene clusters (167 genes) that show higher expression levels in extreme responders.

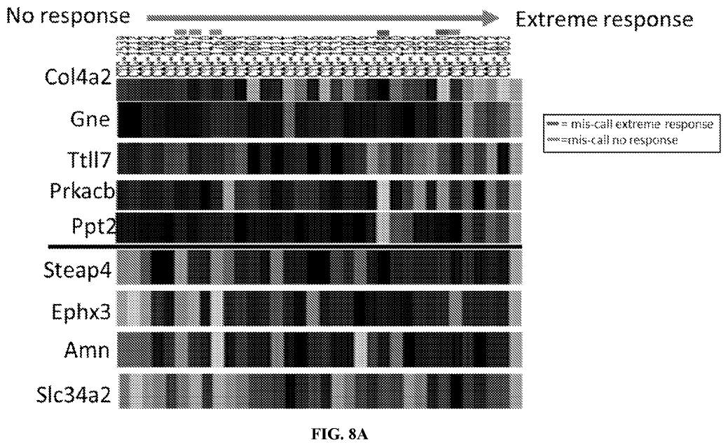

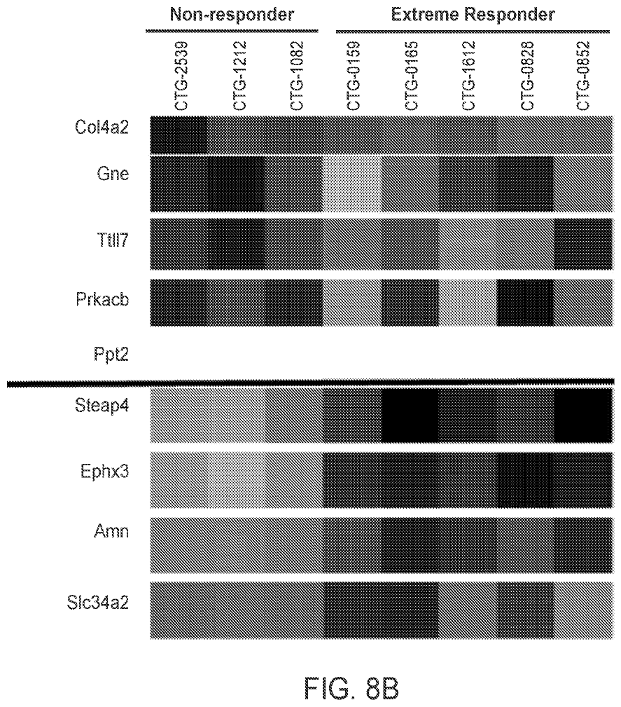

Using RNAseq data, initial analysis of 4 extreme responder models and 3 non-responder models identified a set of 9 genes that are differentially expressed which can be used to predict patient response. Tumors that express low levels of Co14a2, Gne, Tt117, Prkacb, and Ppt2 concurrent with high expression of Steap4, Ephx3, Amn, and S1c34a2 would be predicted to be highly responsive to ULK1 inhibitors. Predicted non-responders display the opposite expression pattern. Using these parameters to score across the 33 models, only 1 model (CTG-0464) is very inaccurately called as a non-responder using this signature. Based on its tumor growth inhibition percentage, this is actually classified a strong responder. Upon further analysis including a fifth extreme responder tumor, this signature holds up relatively well. FIG. 8A shows a heat map of 9 gene signature from original analysis (without full cohort of tumors), and FIG. 8B shows a heat map of 9 gene signature of clusters with all models.

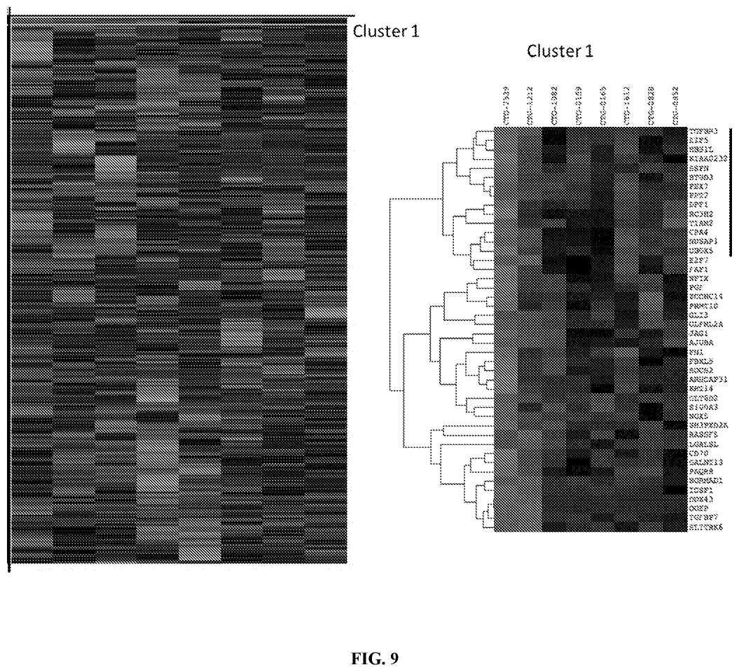

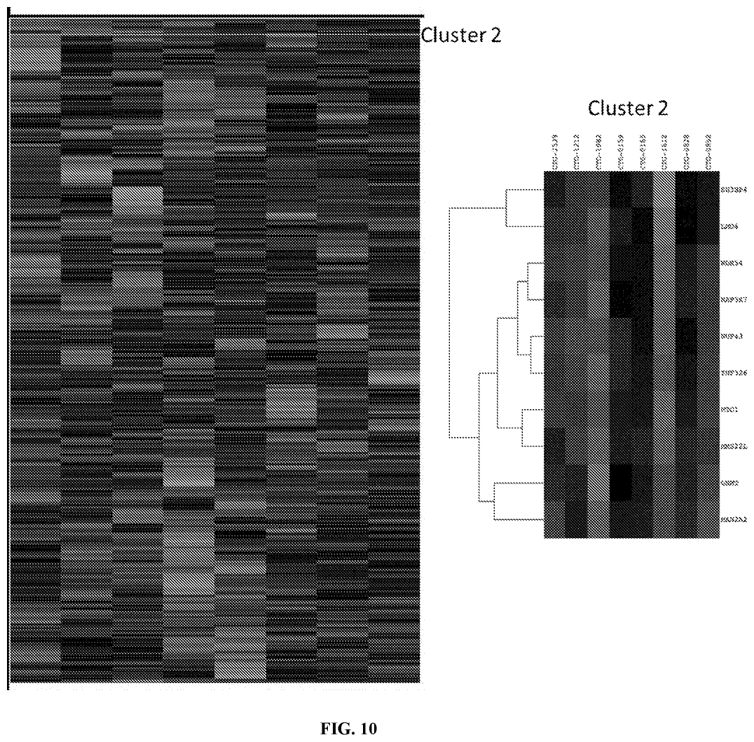

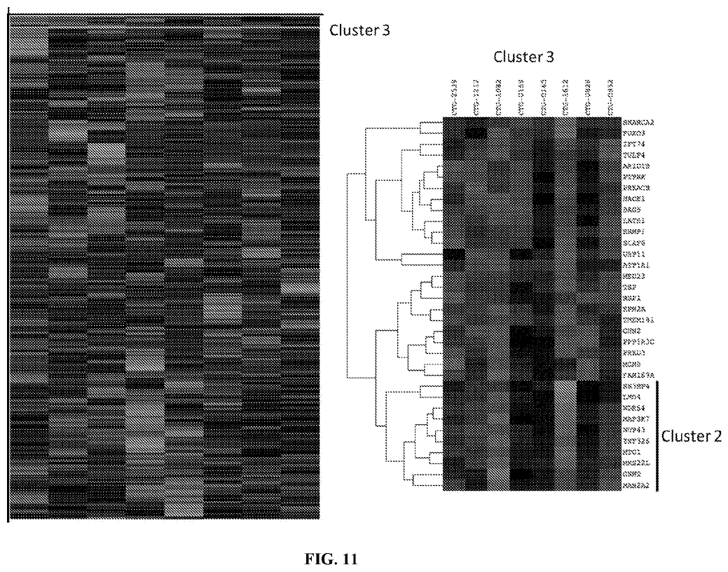

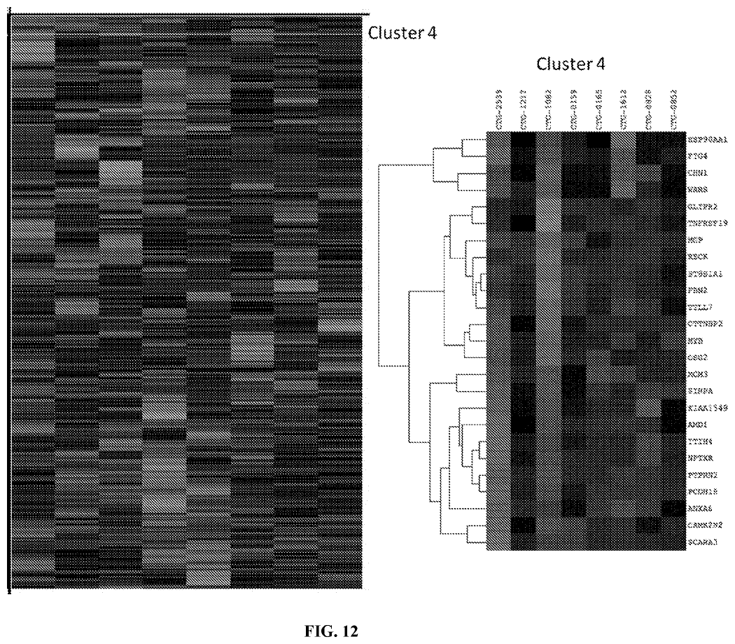

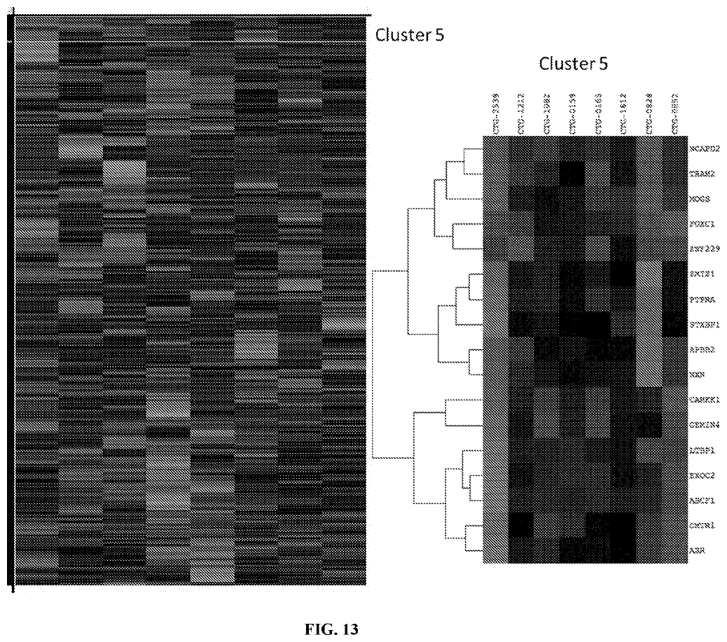

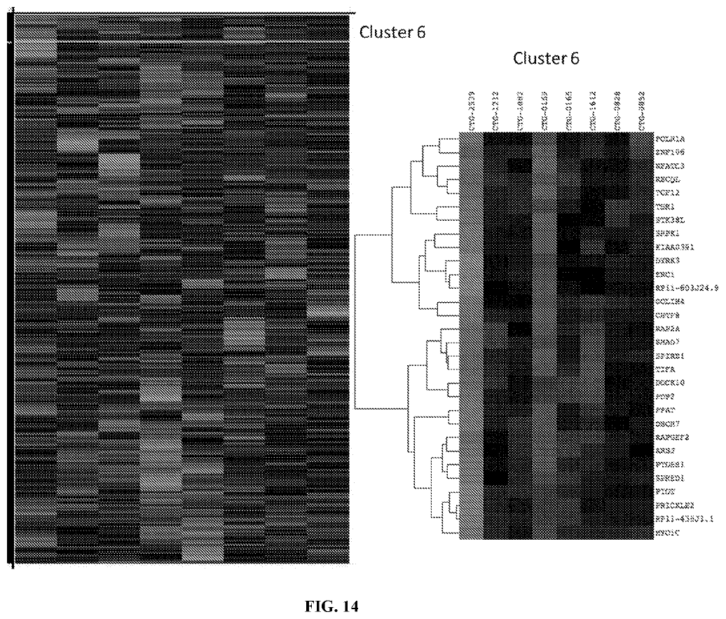

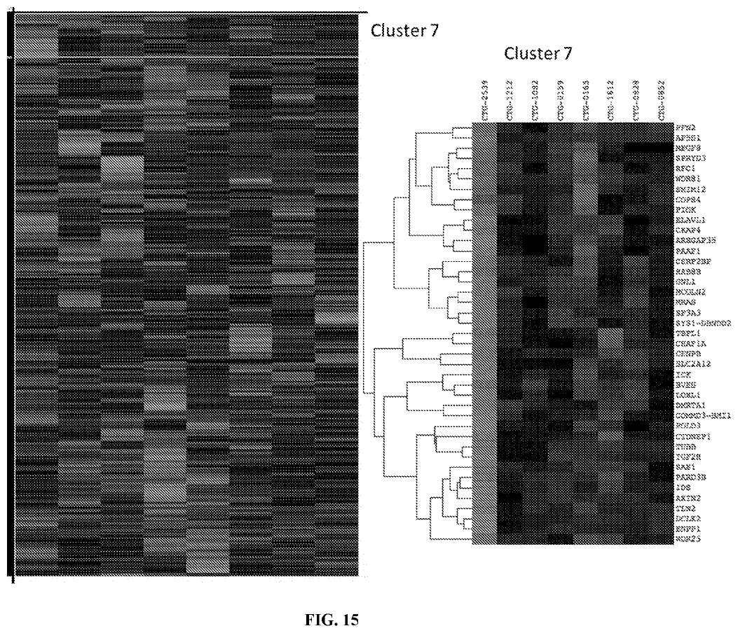

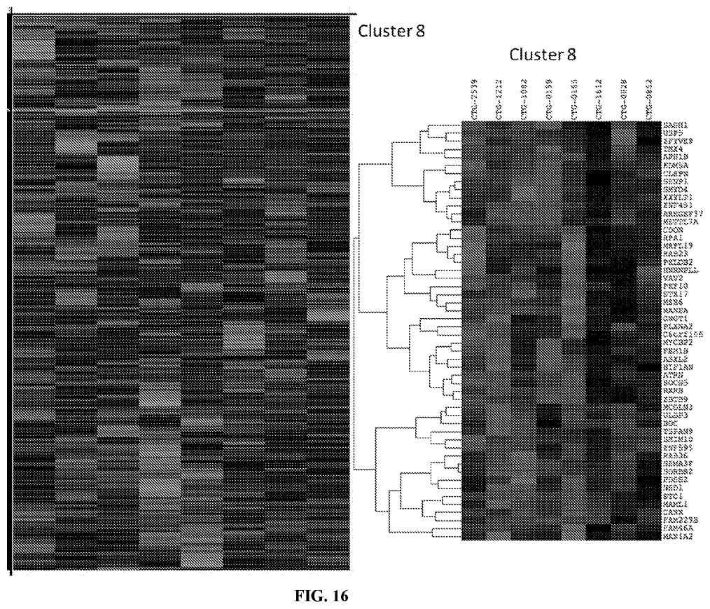

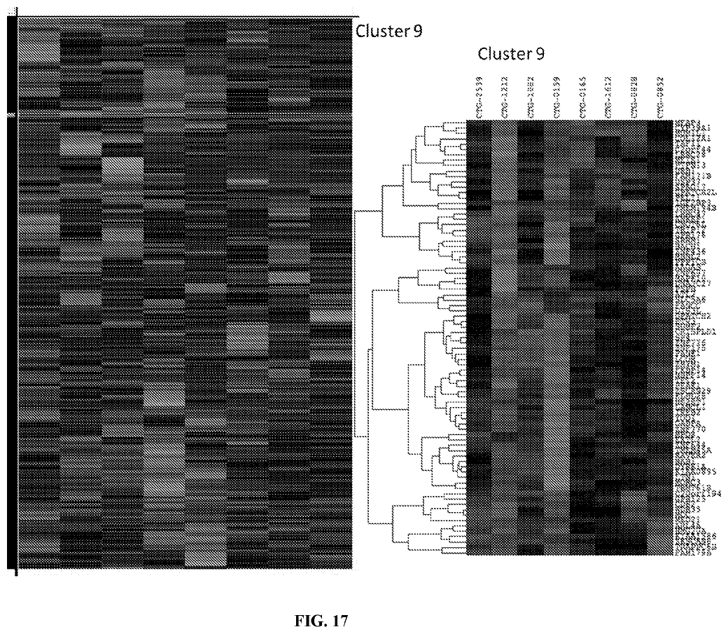

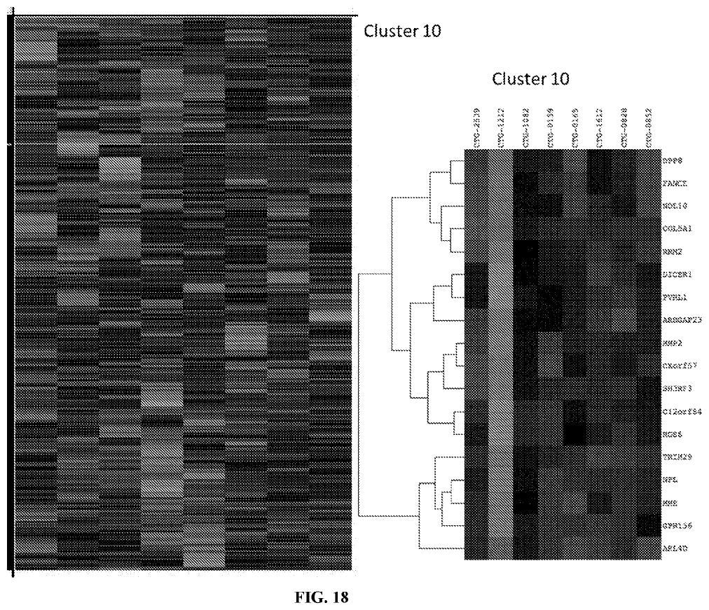

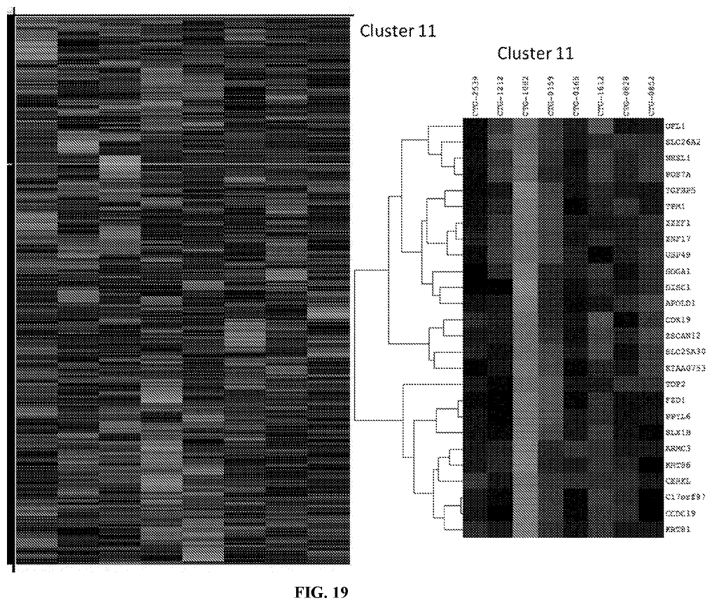

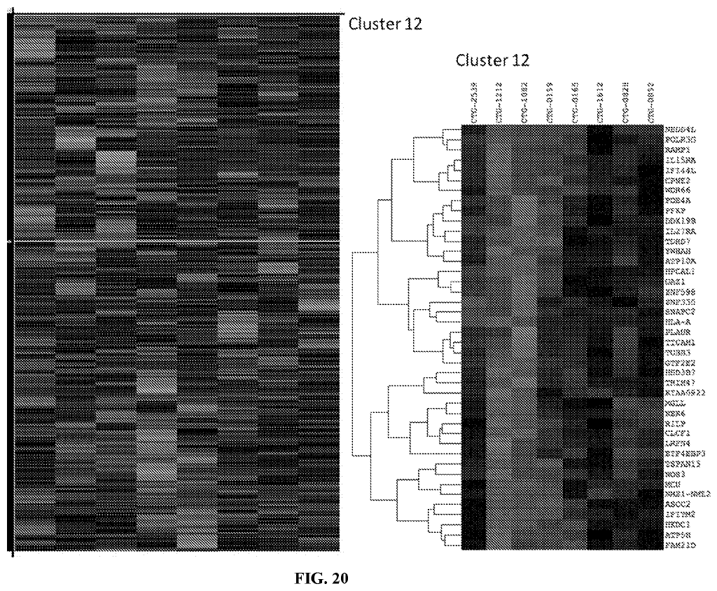

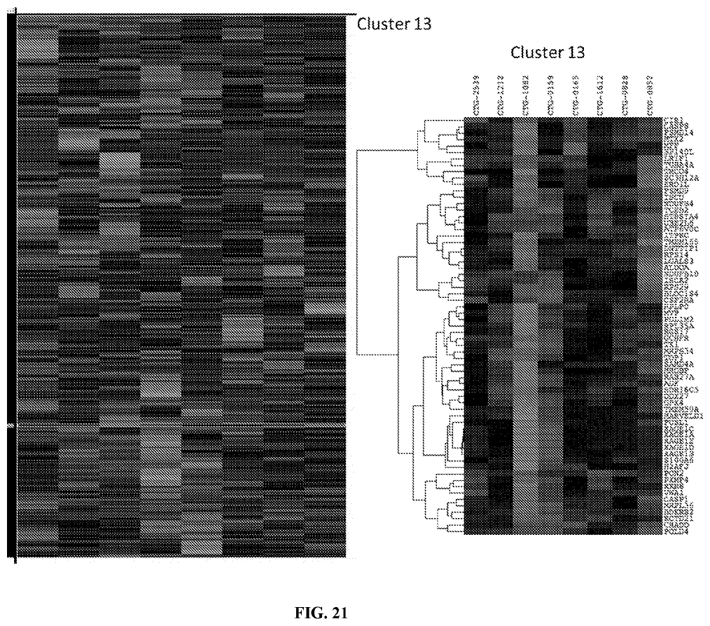

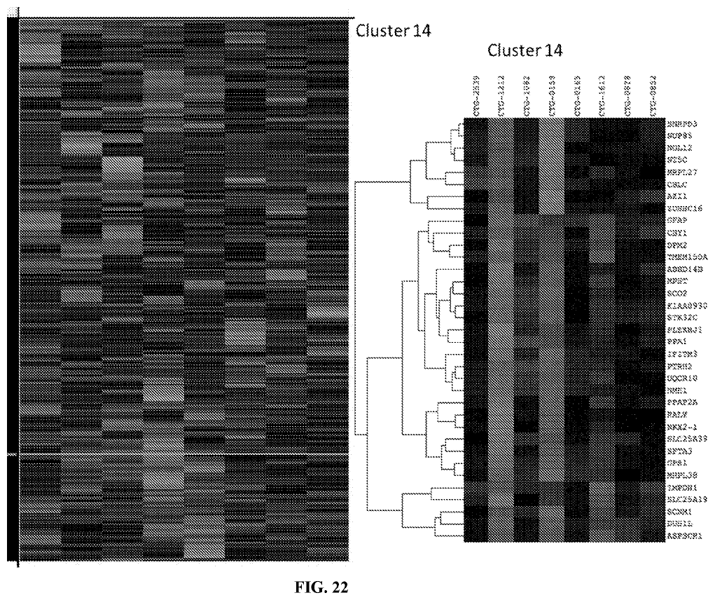

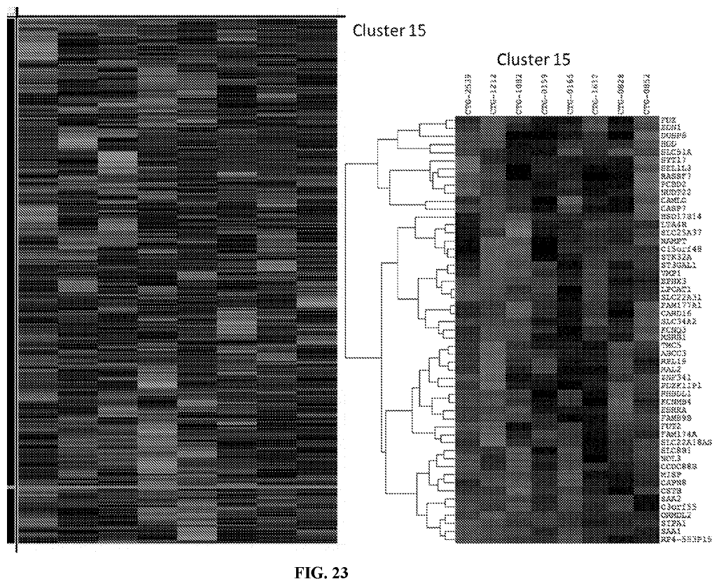

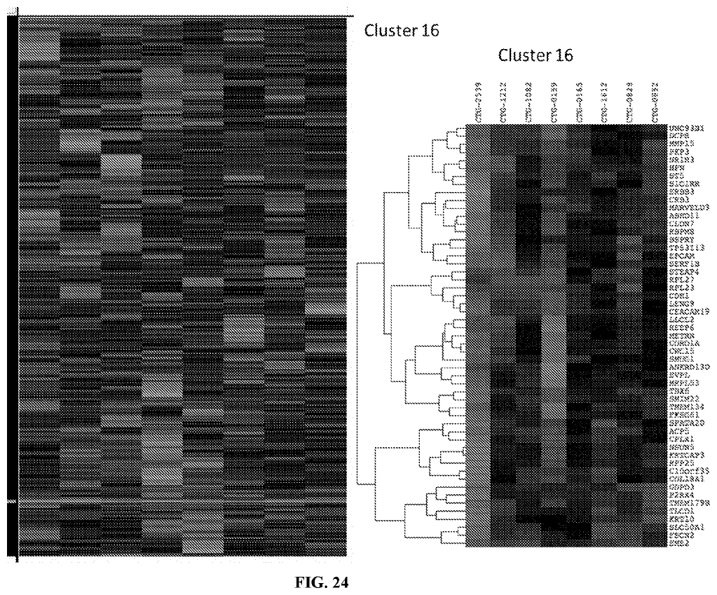

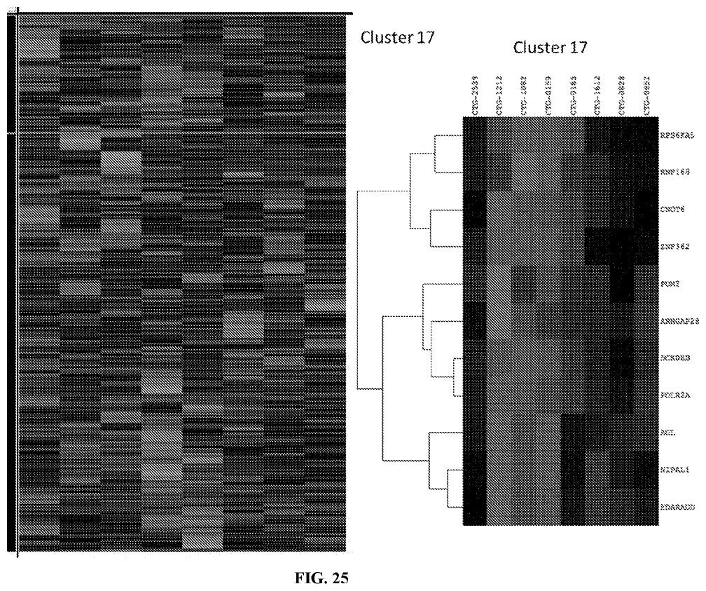

Heat maps of each cluster of genes from cluster 1-17 are shown in FIGS. 9-25.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a disorder mediated by ULK in a subject in need thereof, the method comprising:
administering to the subject a therapeutically effective amount of a ULK inhibitor, wherein a tissue affected by the disorder in the subject has a distinct expression of at least one of biomarker genes in Table 1 or Table 2.

2. A method of predicting a likelihood of success of treating a disorder mediated by ULK with a ULK inhibitor in a subject in need thereof, comprising:
obtaining a gene expression profile of a plurality of genes from a tissue of the subject, wherein the plurality of genes comprises at least one gene in Table 1 or at least one gene in Table 2; and
predicting the likelihood of success of a ULK inhibitor treatment based on the gene profile.

3. A method for selecting a subject for a ULK inhibitor treatment against a disorder mediated by ULK in the patient, comprising:
obtaining a gene expression profile of a plurality of genes from a tissue of the subject, wherein the plurality of genes comprises at least one gene in Table 1 or at least one gene in Table 2; and
selecting the subject for the ULK inhibitor treatment based on the gene profile. cancer.

4. The method of any one of preceding claims, wherein the disorder mediated by ULK is a

5. The method of any one of preceding claims, wherein the tissue is the cancer tissue.

6. The method of claim 1, wherein the distinct expression of at least one of biomarker genes in Table 1 comprises a gene expression level above a predetermined threshold.

7. The method of claim 1, wherein the distinct expression of at least one of biomarker genes in Table 2 comprises a gene expression level below a predetermined threshold.

8. The method of claim 2, wherein the likelihood of success of a ULK inhibitor treatment is predicted high when gene expression level of the at least one gene in Table 1 is above a predetermined threshold.

9. The method of claim 2, wherein the likelihood of success of a ULK inhibitor treatment is predicted high when gene expression levels of the at least two genes in Table 1 are above a predetermined threshold.

10. The method of claim 2, wherein the likelihood of success of a ULK inhibitor treatment is predicted high when gene expression level of the at least one gene in Table 2 is below a predetermined threshold.

11. The method of claim 2, wherein the likelihood of success of a ULK inhibitor treatment is predicted high when gene expression levels of the at least two gene in Table 2 are below a predetermined threshold.

12. The method of claim 3, wherein the subject is selected for the ULK inhibitor treatment when gene expression level of the at least one gene in Table 1 is above a predetermined threshold.

13. The method of claim 3, wherein the subject is selected for the ULK inhibitor treatment when gene expression levels of the at least two genes in Table 1 are above a predetermined threshold.

14. The method of claim 3, wherein the subject is selected for the ULK inhibitor treatment when gene expression level of the at least one gene in Table 2 is below a predetermined threshold.

15. The method of claim 3, wherein the subject is selected for the ULK inhibitor treatment when gene expression levels of the at least two gene in Table 2 are below a predetermined threshold.

16. The method of any one of preceding claims, wherein at least one gene in Table 1 comprises FUZ, EDN1, DUSP8, HGD, SLC51A, SYT17, SEL1L3, RASSF7, PCBD2, NUDT22, CAMLG, CASP7, HSD17B14, LTA4H, SLC25A37, NAMPT, C15orf48, STK32A, or ST3GAL1 .

17. The method of any one of preceding claims, wherein at least one gene in Table 2 comprises SASH1, USP5, ZFYVE0, TMX4, APH1B, KDM5A, CLSPN, SENP1, SMYD4, XXYLT1, ZNF451, ARHGEF37, METTL7A, CDON, RPA1, MRPL19, RAB23, PHLDB2, or HNRNPLL.

18. The method of any one of preceding claims, wherein the ULK inhibitor has a structure of Formula A: 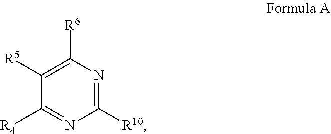
wherein in Formula A:
R¹⁰ is halogen; —OR¹¹, wherein R¹¹ is selected from the group consisting of H, optionally substituted aryl and optionally substituted heteroaryl; —NR¹R², wherein R¹ is H or optionally substituted alkyl and R² is H, optionally substituted aryl, optionally substituted heteroaryl, optionally substituted cycloalkyl, or optionally substituted alkyl, or NR¹R² together form a heterocycle;
R⁴ is optionally substituted amino, optionally substituted aryloxy, optionally substituted heteroaryloxy, optionally substituted alkoxy, N-heterocyclic, optionally substituted thiol, optionally substituted alkyl, hydroxyl or halogen;
or R⁴ and R¹⁰ together form a cyclic structure;
R⁵ is H, hydroxyl, optionally substituted alkyl, halo, optionally substituted alkoxy, or optionally substituted aryl, optionally substituted carboxyl, cyano, or nitro;
or R⁵ and R⁶ together form a cyclic structure; and
R⁶ is H, halogen, or haloalkyl.

19. The method of any one of the preceding claims, wherein the ULK inhibitor is administered as a monotherapy.

20. The method of any one of claims 1-18, wherein the ULK inhibitor is administered to the subject with an additional therapeutic agent.

21. The method of any one of the preceding claims, wherein the cancer is lung cancer, breast cancer, or pancreatic cancer.

22. The method of any one of the preceding claims, wherein the cancer is refractory to a prior treatment.

23. The method of any one of the preceding claims, wherein the cancer is refractory to carboplatin, a carboplatin analog, an MEK inhibitor, trametinib, cobimetinib, binimetinib, selumetinib, erlotinib, gefitinib, osimertinib, crizotinib, pemetrexed, docetaxol, or pembroluzimab.
